(19) United States
(12) Patent Application Publication
(10) Pub. No.: US 2008/0014616 A1
(43) Pub. Date: Jan. 17, 2008

(54) Methods of introducing targeted diversity into nucleic acid molecules

(76) Inventors: Kevin Jarrell, Lincoln, MA (US); Prashanth Vishwanath, Brighton, MA (US); Robert McCarroll, Lexington, MA (US); MIchael Storek, Waltham, MA (US)

Correspondence Address: CHOATE, HALL & STEWART LLP, TWO INTERNATIONAL PLACE, BOSTON, MA, 02110, US

(21) Appl. No.: 11/827,318

(22) Filed: Jul. 11, 2007

Related U.S. Application Data
(60) Provisional application No. 60/830,123, filed on Jul. 11, 2006.

(51) Int. Cl. C12P 19/34

(52) U.S. Cl. 435/91.2

(57) ABSTRACT

Systems are disclosed that are useful for introducing one or more targeted positions or regions of diversity into a nucleic acid molecule. In certain embodiments, diversity in a targeted position or region is generated by providing one or more degenerate primer sets and a template nucleic acid molecule, wherein the primers are extended in opposite directions against the template nucleic acid molecule in a polymerase-mediated extension reaction. In certain embodiments, the generated nucleic acid molecule into which diversity has been introduced comprises single-stranded regions at its termini, which single-stranded regions are capable of annealing to each other.

### CROSS-REFERENCE TO RELATED APPLICATIONS

This application is co-pending with, shares at least one common inventor with, and claims priority to U.S. Provisional Patent Application No. 60/830,123, filed Jul. 11, 2006, the contents of which are hereby incorporated by reference in their entirety.

### BACKGROUND OF THE INVENTION

The molecular biology revolution began with the discovery of enzymes that were capable of cleaving double stranded DNA, so that DNA fragments were produced that could be ligated to one another to generate new, so-called “recombinant” molecules (see, for example, Cohen et al., Proc. Natl. Acad. Sci. USA 70:1293, 1973; Cohen et al., Proc. Natl. Acad. Sci. USA 70:3274, 1973; see also U.S. Pat. Nos. 4,740,470; 4,468,464; 4,237,224). The revolution was extended by the discovery of the polymerase chain reaction (“PCR”), which allowed rapid amplification of particular DNA segments, producing large amounts of material that could subsequently be cleaved and ligated to other DNA molecules (see, for example, U.S. Pat. Nos. 4,683,195; 4,683,202; and 5,333,675).

Further advances in molecular biology have resulted in powerful tools for engineering and recombination of nucleic acids. Restriction enzymes, site-directed mutagenesis, various polymerase-chain-reaction (PCR)-based strategies, synthesis-based strategies, homologous recombination, and other approaches, are all employed in the production of engineered nucleic acids and/or the variation of nucleic acid sequences.

Some of these techniques involve recombination between or among related nucleic acid sequences, typically followed by selection of desired recombined sequences (for example, see Patten et al., U.S. Pat. Nos. 6,579,678 and 6,613,514). Such approaches have significant drawbacks, however, not the least of which is that, due to the stochastic nature of recombination, the practitioner must rely on a chance recombination event to generate a particular nucleic acid sequence. Furthermore, one or more of the parental molecules may fail to undergo recombination or may be reconstituted in a recombination reaction, such that extensive screening is required to identify new recombinants of interest.

Despite the utility of these and other techniques for generating engineered nucleic acid sequences and for generating diversity in a population of nucleic acid molecules, there remains substantial room for improvement.

### SUMMARY OF THE INVENTION

Systems and methods of the present invention are useful for introducing diversity into one or more targeted positions or regions of a nucleic acid molecule. In certain embodiments, diversity is targeted to one or more positions or regions by providing two primers and a template nucleic acid molecule, wherein the primers are extended in opposite directions against a template nucleic acid molecule in a polymerase-mediated extension reaction. In certain embodiments, variant nucleic acid molecules into which diversity has been introduced include single-stranded regions at their termini, which single-stranded regions are capable of annealing to each other under at least one set of annealing conditions.

In certain embodiments, one or both of the primers comprise a degenerate primer set that contains one or more degenerate positions such that the polymerase-mediated extension reaction generates a plurality of variant nucleic acid molecules, wherein the diversity present in the plurality of variant nucleic acid molecules reflects the diversity present in the degenerate primer set. In certain embodiments, one or more degenerate positions in the degenerate primer set are fully degenerate. For example, in the context of naturally occurring nucleotide residues, a primer residue is fully degenerate when one or more degenerate position or positions are represented by the four naturally-occurring nucleotide bases: adenine, cytosine, guanine and thymidine/uracil (depending on whether the primer comprises deoxyribonucleotide or ribonucleotide residues). Additionally or alternatively, one or more degenerate positions in the degenerate primer set may be fully degenerate when the degenerate position or positions are represented by non-naturally occurring nucleotide bases. In certain embodiments, a fully degenerate primer position comprises both naturally occurring and non-naturally occurring nucleotide bases. In certain embodiments, one or more degenerate positions are less than fully degenerate. For example, in the context of naturally occurring nucleotide residues, degenerate position or positions may be represented by fewer than the four naturally-occurring nucleotide bases. Thus, in certain embodiments, a degenerate position comprises two or three alternative nucleic acid residues that contain a base selected from the group consisting of adenine, cytosine, guanine, and thymidine/uracil. Additionally or alternatively, a less than fully degenerate position may comprise one or more nucleotides that do not occur naturally.

In certain embodiments, at least one of the primers used in the polymerase-mediated extension contains a terminator nucleotide that does not serve as a replication template for the polymerase used in the extension reaction under at least one set of reaction conditions, such that members of the generated plurality of variant nucleic acid molecules contain at least one overhang. As non-limiting examples, the terminator nucleotide may be a ribonucleotide or a 2′-O-methyl nucleotide. In certain embodiments, a terminator nucleotide may be capable of being used as a template by a different polymerase. Additionally or alternatively, a terminator nucleotide may capable of being be used as a template by the same polymerase under a different set of reaction conditions. In certain embodiments, one or both primers contain more than one terminator residue. In certain embodiments, one or both primers contain two or more different types of terminator residues.

In some embodiments, at least one of the primers used in the polymerase-mediated extension reaction contains a terminator structure that does not serve as a replication template for a polymerase used in the extension reaction. In some embodiments, a terminator structure may serve as a replication template for a polymerase used in the extension reaction and/or the polymerase may be able to read through the terminator structure. In certain embodiments, both primers used in a polymerase-mediated extension contain a terminator nucleotide and/or a terminator structure. In certain embodiments, a terminator structure is an abasic site.

In certain embodiments, degenerate position(s) of one or both primers are located 5′ to a terminator nucleotide or structure. According to such embodiments, after polymerase-mediated extension, degenerate position(s) will be located in the single-stranded 5′ overhangs of a generated plurality of variant nucleic acid molecules. Additionally or alternatively, degenerate position(s) of one or both primers may be located 3′ to the terminator nucleotide or structure, provided that the degenerate position(s) do not prevent the primer from annealing to a template nucleic acid molecule and priming extension. According to such embodiments, after polymerase-mediated extension, degenerate position(s) will be located in the double-stranded regions of a generated plurality of variant nucleic acid molecules.

In certain embodiments, at least one of the primers used in the polymerase-mediated extension contains one or more nucleotide residues that are capable of being cleaved or removed under certain conditions. For example, the primer may contain one or more ribonucleotide residues. After extension by a polymerase that is able to use ribonucleotide residues as templates, the ribonucleotide residue(s) may be cleaved or removed from the extension product by any of a number of methods well known in the art. For example, ribonucleotide residues may be cleaved or removed by exposure to elevated pH (e.g., treatment with a base such as sodium hydroxide). Any other treatment that cleaves or removes ribonucleotide residues without disturbing DNA residues (e.g., exposure to RNase, etc.) could alternatively be employed at this step. One of ordinary skill in the art will be aware of other known treatments that cleave or remove ribonucleotide or other cleavable/removable residues such that the treated molecule may be used in accordance with systems and methods disclosed herein. In certain embodiments, the region that is capable of being cleaved or removed extends to the 5′ end of one or both primers. In such embodiments, removal of the region(s) after polymerase-mediated extension results in a partially double-stranded extension product that contains at least one terminal 3′ single-stranded overhang.

In certain embodiments, degenerate position(s) of one or both primers are located in the region that is capable of being cleaved or removed after extension. According to such embodiments, after polymerase-mediated extension and cleavage or removal of the region, degenerate position(s) will be located in the 3′ overhangs of the plurality of variant nucleic acid molecules. Additionally or alternatively, degenerate position(s) of one or both primers may be located 3′ to the region to that is capable of being cleaved or removed after extension, provided that the degenerate position(s) do not prevent the primer from annealing to a template nucleic acid molecule and priming extension. According to such embodiments, degenerate position(s) will be located in the double-stranded regions of the plurality of variant nucleic acid molecules.

In certain embodiments, the polymerase-mediated extension reaction is performed in accordance with any of the teachings of U.S. Pat. No. 6,358,712, U.S. patent application Ser. No. 10/272,351 and/or U.S. patent application Ser. No. 10/383,135, each of which is incorporated herein by reference in its entirety.

In certain embodiments, variant nucleic acid molecules into which one or more targeted positions or regions of diversity have been introduced include single-stranded regions at their termini, which single-stranded regions are at least partially complementary and capable of annealing to each other. In certain embodiments, mismatches are created upon annealing such single-stranded regions. For example, a degenerate primer set may be used to introduce targeted diversity into one or more positions or regions of a nucleic acid molecule via polymerase-mediated extension, generating a plurality of variant nucleic acid molecules. Each member of the plurality of variant nucleic acid molecules may include single-stranded regions at its termini, which single-stranded regions are not perfectly complementary, but are nevertheless capable of annealing. When such single-stranded termini are annealed, the annealed region will contain mismatches at the positions where the termini are not perfectly complementary.

In certain embodiments, systems of the present invention are useful for introducing diversity into two or more targeted positions or regions of a nucleic acid molecule by subjecting a template nucleic acid molecule to two or more simultaneous or sequential polymerase-mediated extension reactions. For example, a template nucleic acid molecule may be subjected to two or more polymerase-mediated extension reactions using two or more pairs of degenerate primer sets to generate multiple pluralities of variant nucleic acid molecules, each of the pluralities corresponding to a portion of the template nucleic acid molecule and each containing one or more targeted positions or regions of diversity. In certain embodiments, individual members of each of the pluralities of variant nucleic acid molecules are capable of annealing to individual members of one or more of the other pluralities to generate a new plurality of variant nucleic acid molecules, members of which comprise targeted regions of diversity at two or more positions. Members of such a new plurality of variant nucleic acid molecules may be used as template nucleic acid molecules for subsequent introduction of further diversity into the same or different positions or regions.

In certain embodiments, a template nucleic acid molecule is subjected to two or more iterative polymerase-mediated extension reactions. In some embodiments, iterative polymerase-mediated extension reactions may be used to generate diversity in a single position or region of a template nucleic acid molecule. For example, a first plurality of variant nucleic acid molecules that contains diversity within a single targeted position or region may be generated. One or more members of the first plurality may then be used as template nucleic acid molecules in one or more subsequent polymerase-mediated extension reactions to generate a new plurality of variant nucleic acid molecules that contains further diversity in the originally targeted position or region. Such a first plurality of variant nucleic acid molecules may optionally be tested or screened and one or more members of the first plurality with one or more desirable properties may be selected for use as template nucleic acid molecules in the subsequent extension reaction(s). This process may be repeated to generate further diversity in the targeted position or region.

Additionally or alternatively, iterative polymerase-mediated extension reactions may be used to create diversity in two or more positions or regions of a template nucleic acid molecule. For example, a first plurality of variant nucleic acid molecules that contains diversity within a single targeted position or region may be generated. One or more members of the first plurality may then be used as template nucleic acid molecules in one or more subsequent polymerase-mediated extension reactions to generate a new plurality of variant nucleic acid molecules that contains further diversity in one or more additional targeted positions or regions. Such a first plurality of variant nucleic acid molecules may optionally be tested or screened and one or more members of the first plurality with one or more desirable properties may be selected for use as template nucleic acid molecules in the subsequent extension reaction(s). This process may be repeated to generate further diversity in multiple targeted positions or regions.

In certain embodiments, a template nucleic acid molecule is or comprises a vector. For example, a template nucleic acid molecule may be a plasmid, a yeast artificial chromosome (“YAC”), a bacterial artificial chromosome (“BAC”) or a phage vector. A vector template nucleic acid molecule may either be circular or linear.

In certain embodiments, a template nucleic acid molecule is a naturally occurring molecule. For example, a template nucleic acid molecule may be isolated from or found in the genome of a particular organism or cell. Alternatively, a template nucleic acid molecule may be derived from the nucleotide sequence of an mRNA expressed in a particular organism or cell, for example by generating a cDNA. In certain embodiments, a template nucleic acid molecule is the expressed mRNA itself. For example, mRNA may be isolated from a cell or organism of interest and subjected to polymerase-mediated extension reaction using a polymerase that is capable of using ribonucleotide residues as templates. In certain embodiments, the polymerase used is capable of using both ribonucleotide residues and deoxyribonucleotide residues as templates.

In certain embodiments, a template nucleic acid molecule is a molecule that does not occur naturally. For example, a template nucleic acid molecule may comprise two or more naturally occurring sequences, which sequences are not naturally present in the same nucleic acid molecule. Additionally or alternatively, a template nucleic acid molecule may comprise two or more naturally occurring sequences, which sequences are present in the same nucleic acid molecule but are present in a positional relationship different from that in which they occur in the template nucleic acid molecule. Additionally or alternatively, a template nucleic acid molecule may comprise one or more artificial nucleic acid sequences that do not occur naturally in any organism. In certain embodiments, a template nucleic acid molecule may comprise both naturally and non-naturally occurring nucleic acid sequences. In some embodiments, a template nucleic acid molecule is non-natural in that it is created by the hand of man. In some embodiments, a non-natural template nucleic acid molecule is created by an automated or semi-automated robotic or other mechanical process.

In certain embodiments, a template nucleic acid molecule is created by any of the methods described in U.S. patent application Ser. No. 11/271,561, incorporated herein by reference in its entirety. For example, a template nucleic acid molecule may be generated by building a ladder complex of partially complementary oligonucleotides. A ladder complex generated according to any of the methods described in the Ser. No. 11/271,561 application may be converted into an unnicked double-stranded or partially double-stranded template nucleic acid molecule prior to introducing diversity into one or more targeted positions or regions using any of the systems described herein. Alternatively, a ladder complex generated according to any of the methods described in the Ser. No. 11/271,561 application may be converted into an unnicked double-stranded or partially double-stranded product molecule while simultaneously introducing diversity into one or more targeted positions or regions of a nucleic acid molecule using any of the systems described herein.

In certain embodiments, variant nucleic acid molecules generated by systems disclosed herein encode polypeptide sequences. In certain embodiments, systems of the present invention are used to produce a plurality of variant nucleic acid molecules that encode a plurality of polypeptides containing one or more targeted positions or regions of amino acid sequence diversity. In certain embodiments, systems of the present invention may be used to perform saturation mutagenesis on a polypeptide of interest. For example, systems of the present invention may be used to produce a plurality of variant nucleic acid molecules that encode a plurality of polypeptides containing all possible amino acid substitutions at one or more amino acid positions. In certain embodiments, systems of the present invention may be used to produce a plurality of variant nucleic acid molecules that encode a plurality of polypeptides that contain a functional domain or a portion of a functional domain of interest, which domain or portion contains one or more targeted positions or regions of sequence diversity. In certain embodiments, systems of the present invention may be used to generate one or more point mutations, deletions, insertions or rearrangements in the functional domain or portion such that one or more functions of the encoded polypeptide are enhanced, decreased, or otherwise altered.

In certain embodiments, a plurality of variant nucleic acid molecules generated according to one or more systems disclosed herein comprises a library of nucleic acid molecules. In certain embodiments, phylogenetic analysis is used to guide the design of the degenerate primer set used in a template-mediated polymerization reaction such that the diversity introduced into one or more targeted positions or regions of a template nucleic acid molecule is restricted to only particular nucleotide positions and/or only a subset of the possible nucleotides are encoded at those positions. In certain embodiments, members of the library of nucleic acid molecules encode polypeptides.

In certain embodiments, systems of the present invention are used to introduce diversity into one or more targeted positions or regions of a non-coding nucleic acid molecule. For example, systems of the present invention can be used to introduce or alter a regulatory element that regulates the expression of a polypeptide of interest. In some embodiments, a promoter region or element can be introduced or altered according to certain systems of the present invention to determine which residues of the promoter region or element are important for directing expression of a polypeptide under control of that promoter or element. In some embodiments, systems of the present invention can be used to introduce an otherwise heterologous promoter element into a promoter that lacks that element. For example, a tissue specific or inducible control element may be introduced into a constitutive promoter. In some embodiments, systems of the present invention can be used to introduce an intron or splicing site into a nucleic acid of interest that encodes a polypeptide, or alter an existing intron or splicing site. In some embodiments, systems of the present invention can be used to introduce a regulatory element into the 3′ or 5′ untranslated region (“UTR”) of a particular mRNA molecule, or alter an existing 3′ or 5′ UTR regulatory element.

### BRIEF DESCRIPTION OF THE DRAWING

FIG. 1 shows one embodiment of introducing targeted diversity into a nucleic acid molecule using a degenerate primer set and polymerase-mediated extension. The arrows on the primers of the illustrated degenerate primer set indicate 5′ to 3′ orientation. Terminator residues are depicted by filled circles. Degenerate positions are depicted by open circles and squares. Upon polymerase-mediated extension and self-annealing, a plurality of circular double-stranded molecules containing two nicks is generated.

FIG. 2 shows one embodiment of introducing targeted diversity into multiple regions of a nucleic acid molecule using a degenerate primer set and polymerase-mediated extension. The arrows on the primers of the illustrated degenerate primer set indicate 5′ to 3′ orientation. Terminator residues are depicted by filled circles. Degenerate positions are depicted by open circles, squares and triangles. In the embodiment shown, diversity is targeted to two regions of the template nucleic acid molecule by performing two polymerase-mediated extension reactions. However, one of ordinary skill in the art will understand that more than two regions may be targeted using more than two polymerase-mediated extension reactions. The polymerase-mediated extension reactions may be performed sequentially or simultaneously. Upon polymerase-mediated extension and annealing, a plurality of circular double-stranded molecules containing multiple nicks is generated.

FIG. 3 shows one embodiment of introducing targeted diversity into a nucleic acid molecule using a degenerate primer set and polymerase-mediated extension wherein an additional residue that does not correspond in position to a residue in the template nucleic acid molecule is introduced. The arrows on the primers of the illustrated degenerate primer set indicate 5′ to 3′ orientation. Terminator residues are depicted by filled circles. Degenerate positions are depicted by open circles and squares. Residues to be added are depicted by filled stars. Upon polymerase-mediated extension and self-annealing, a plurality of circular double-stranded molecules containing two nicks and the added residue is generated.

FIG. 4 shows one embodiment of introducing targeted diversity into a nucleic acid molecule using a degenerate primer set and polymerase-mediated extension wherein a residue in the template nucleic acid molecule is deleted. The arrows on the primers of the illustrated degenerate primer set indicate 5′ to 3′ orientation. Terminator residues are depicted by filled circles. Degenerate positions are depicted by open circles and squares. The residue to be deleted is depicted by open brackets. Upon polymerase-mediated extension and self-annealing, a plurality of circular double-stranded molecules containing two nicks but lacking the deleted residue is generated.

FIG. 5 shows one embodiment of introducing targeted diversity into a nucleic acid molecule using polymerase-mediated extension and a degenerate primer set that contain regions that are capable of being cleaved or removed. The arrows on the primers of the illustrated degenerate primer set indicate 5′ to 3′ orientation. Brackets indicated cleavable/removable regions. Degenerate positions are depicted by open circles and squares. In the embodiment shown, the degenerate positions are within the regions that are capable of being cleaved or removed. Upon cleavage or removal and self-annealing, a plurality of circular double-stranded molecules containing two nicks is generated.

FIG. 6 shows one embodiment in which a plurality of variant nucleic acid molecules generated by a polymerase-mediated extension reaction using a degenerate primer set is combined with a recipient nucleic acid molecule. The arrows on the primers of the illustrated degenerate primer set indicate 5′ to 3′ orientation. Terminator residues are depicted by filled circles. Degenerate positions are depicted by open circles, squares and triangles. Representative members of the plurality of variant nucleic acid molecules and the plurality of variant nucleic acid molecules annealed to recipient nucleic acid molecules are depicted inside the large brackets; ellipses indicate that not all members of the pluralities are shown. Upon combining the plurality of variant nucleic acid molecules with the recipient nucleic acid molecule, a plurality of circular double-stranded molecules containing multiple nicks is generated.

FIG. 7 shows one embodiment in which a plurality of variant nucleic acid molecules generated by a polymerase-mediated extension reaction using a degenerate primer set is combined with a recipient nucleic acid molecule in a non-directional manner. The arrows on the primers of the illustrated degenerate primer set indicate 5′ to 3′ orientation. Terminator residues are depicted by filled circles. Degenerate positions are depicted by open circles, squares and triangles. Representative members of the plurality of variant nucleic acid molecules and the plurality of variant nucleic acid molecules annealed to recipient nucleic acid molecules are depicted inside the large brackets; ellipses indicate that not all members of the pluralities are shown. Upon combining the plurality of variant nucleic acid molecules with the recipient nucleic acid molecule, a plurality of circular double-stranded molecules containing multiple nicks is generated, wherein variant nucleic acid molecules are combined in both orientations with a recipient nucleic acid molecule.

FIG. 8 shows one embodiment in which a plurality of variant nucleic acid molecules generated by a polymerase-mediated extension reaction using a degenerate primer set is combined with multiple recipient nucleic acid molecules. The arrows on the primers of the illustrated degenerate primer set indicate 5′ to 3′ orientation. Terminator residues are depicted by filled circles. Degenerate positions are depicted by open circles, squares and triangles. Representative members of the plurality of variant nucleic acid molecules and the plurality of variant nucleic acid molecules annealed to recipient nucleic acid molecules are depicted inside the large brackets; ellipses indicate that not all members of the pluralities are shown. Upon combining the plurality of variant nucleic acid molecules with multiple recipient nucleic acid molecules, a plurality of circular double-stranded molecules containing multiple nicks is generated.

### DEFINITIONS

“Corresponding Position”, “Correspond in Position”: The terms “corresponding position” and “correspond in position,” as used herein, refer to a nucleotide or amino acid residue in a nucleic acid molecule or polypeptide that is located at the same relative position along the length of a substantially similar or homologous nucleic acid molecule or polypeptide as another nucleotide or amino acid residue. For example, where two nucleic acid molecules are of the same length and sequence but for a difference in sequence at a single nucleotide residue, the differing nucleotide residues correspond in position. Similarly, where two nucleic acid molecules are largely identical in length and sequence but differ in sequence at multiple nucleotide residues, the differing nucleotide residues correspond in position. Where two nucleic acid molecules or polypeptides of otherwise similar or identical sequence contain one or more deletions, insertions, substitutions or rearrangements as compared to each other, two nucleotide or amino acid residues correspond in position when they are located at the same relative position along the length of the nucleic acid molecules or polypeptides beginning at the point where the deletion, insertion, substitution or rearrangement ends. Where a first nucleic acid molecule or polypeptide contains one or more additional nucleotide or amino acid residues as compared to a second substantially similar nucleic acid molecule or polypeptide, the additional nucleotide or amino acid residues present in the first nucleic acid molecule or polypeptide do not correspond in position to any nucleotide or amino acid residues of the second nucleic acid molecule or polypeptide. Corresponding nucleotide or amino acid residues need not possess similar physical properties. For example, a hydrophobic amino acid may correspond in position to a hydrophilic amino acid. Similarly, a purine nucleotide may correspond in position to a pyrimidine nucleotide. One of ordinary skill in the art will be able to recognize where a nucleotide or amino acid residue corresponds in position to another nucleotide or amino acid residue based on any number of recognizable indicia including, but not limited to the sequence identity or similarity of the two nucleic acid molecules or polypeptides. In some embodiments, corresponding positions are identified through the use of an algorithm or computer program. For example BLASTN, BLASTP, Gapped BLAST, etc., may be used to generate alignments and to identify corresponding positions between one or more sequences of interest and/or between one or more sequences of interest and sequences in any of a variety of public databases. The algorithm of Karlin and Altschul (Karlin and Altschul, Proc. Natl. Acad. Sci. USA 87:22264-2268, 1990) modified as in Karlin and Altschul (Proc. Natl. Acad. Sci. USA 90:5873-5877, 1993) is incorporated into the NBLAST and XBLAST programs of Altschul et al. (Altschul, et al., J. Mol. Biol. 215:403-410, 1990). To obtain gapped alignments for comparison purposes, Gapped BLAST may be utilized as described in Altschul et al. (Altschul, et al. Nucleic Acids Res. 25: 3389-3402, 1997). When utilizing BLAST and Gapped BLAST programs, default parameters of the respective programs may be used. Alternatively, the practitioner may use non-default parameters depending on his or her experimental and/or other requirements. See the Web site having URL www.ncbi.nlm.nih.gov.

“Degenerate”, “Degeneracy”: The terms “degenerate” and “degeneracy”, as used herein when referring to a primer or other nucleic acid molecule, refer to the condition of comprising two or more alternative nucleotide residues at a corresponding position in a plurality of primers or nucleic acid molecules. The concept of degeneracy will be familiar to those of ordinary skill in the art. In certain embodiments, diversity is introduced into one or more targeted positions or regions of a nucleic acid molecule through the use of a degenerate primer set in a polymerase-mediated extension reaction. For example, one or both primers may include one or more degenerate positions, such that polymerase-mediated extension of a template nucleic acid molecule generates a plurality of variant nucleic acid molecules. As will be clear from context, “degenerate” and “degeneracy” also refer to the condition of comprising two or more amino acid residues at a corresponding position in a plurality of polypeptide sequences. A nucleic acid molecule or polypeptide may contain one or more positions that are completely degenerate. For example, a position is completely degenerate when every possible naturally occurring nucleotide or amino acid residue occurs at a given position. In certain embodiments, a completely degenerate position comprises residues that do not occur naturally. Additionally or alternatively, an oligonucleotide, nucleic acid molecule or polypeptide may contain one or more positions that are less than completely degenerate. For example, a degenerate nucleotide position may be represented by only two or three of the naturally occurring nucleotides. In certain embodiments, a less than completely degenerate position comprises residues that do not occur naturally. Alternatively, a given amino acid position may be represented by less than the twenty naturally occurring amino acid residues. In certain embodiments, degenerate positions of a nucleic acid molecule or polypeptide may comprise one or more residues that are do not occur naturally.

“Degenerate Primer Set”: The term “degenerate primer set” as used herein, refers to a set of primers that contain one or more positions or regions of degeneracy. In certain embodiments, a degenerate primer set is used in a polymerase-mediated extension reaction to generate a plurality of variant nucleic acid molecules. In certain embodiments, a degenerate primer set used in conjunction with a non-degenerate primer in a polymerase-mediated extension reaction. In such embodiments, a plurality of variant nucleic acid molecules is generated that comprises one or more degenerate positions or regions that correspond to the degenerate positions or regions of the degenerate primer set. In certain embodiments, a degenerate primer set used in conjunction with another degenerate primer set in a polymerase-mediated extension reaction. In such embodiments, a plurality of variant nucleic acid molecules is generated that comprises one or more degenerate positions or regions that correspond to the degenerate positions or regions of both of the degenerate primer sets.

“Naturally Occurring”: The term “naturally occurring”, as used herein when referring to a deoxyribonucleotide residue, refers to one of the four nucleotides containing the bases adenine, cytosine, guanine and thymidine. The term “naturally occurring”, as used herein when referring to a ribonucleotide residue, refers to one of the four nucleotides containing the bases adenine, cytosine, guanine and uracil. As will be clear to one of ordinary skill in the art, the term “naturally occurring” encompasses nucleotides that have been modified as they are naturally modified in a cellular environment. For example, 5-methylcytosine is a natural modification of cytosine bases that certain cells use to mark parental DNA strands during the synthesis of new DNA strands. One of ordinary skill in the art will be aware of other natural modifications to nucleotides. The term “naturally occurring”, as used herein when referring to an amino acid, refers to one of the standard group of twenty amino acids that are the building blocks of polypeptides of most organisms, including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, the term “naturally occurring” also refers to amino acids that are used less often and are not included in this standard group of twenty but are still used by organisms and incorporated into certain polypeptides. For example, the codons UAG and UGA normally encode stop codons in most organisms. However, in some organisms the codons UAG and UGA encode the amino acids selenocysteine and pyrrolysine. Thus, in certain embodiments, selenocysteine and pyrrolysine are naturally occurring amino acids. The term “naturally occurring”, as used herein when referring to a nucleic acid sequence, refers to a nucleic acid sequence that is found in at least one cell or organism. As non-limiting examples, genomic or organellar DNA or RNA, expressed mRNAs (whether fully or incompletely processed), incompletely spliced mRNAs, ribosomal RNAs, transfer RNAs, or snRNAs are all naturally occurring sequences. The present invention encompasses the use of both naturally occurring and non-naturally occurring nucleotide residues and amino acids. One of ordinary skill in the art will understand that even though a nucleotide or amino acid residue is not naturally occurring, such a residue may nevertheless be used in systems of the present invention. For example, a non-naturally occurring nucleotide or amino acid residue may be incorporated into a nucleic acid strand or amino acid chain. In certain embodiments, non-naturally occurring nucleotide and amino acid residues are used to target one or more positions or regions of diversity into a nucleic acid molecule.

“Overhang”: The term “overhang” as used herein, refers to a terminal single-stranded region extending from a double-stranded region of a nucleic acid molecule. In certain embodiments, a nucleic acid molecule comprises a central double-stranded region from which extend two single-stranded overhangs.

“Primer”: The term “primer”, as used herein, refers to an oligonucleotide that is characterized by an ability to be extended against a template nucleic acid molecule, so that a polynucleotide strand whose sequence is complementary to that of at least a portion of the template molecules, is produced linked to the primer. Primers may be of any convenient length selected by the practitioner so long as they are able to anneal to and be extended against a template nucleic acid molecule. For example, the primers of the present invention may be 5, 6, 7, 8, 9, 10, 11, 12, 13, 14, 15, 16, 17, 18, 19, 20, 25, 30, 35, 40, 45, 50 or more nucleotides in length. In certain embodiments, one or more primers that are extended against a template nucleic acid molecule to introduce one or more targeted positions or regions of diversity contain one or more terminator nucleotides or terminator structures that cannot be copied by the polymerase used in the extension reaction under the conditions of the reaction. In certain embodiments, one or more terminator nucleotides present in the primer can be copied by a different polymerase and/or by the same polymerase under different extension conditions. In certain embodiments, one or more primers that are extended against a template nucleic acid molecule to introduce one or more targeted positions or regions of diversity contain one or more residues that may be cleaved or removed subsequent to the polymerase-mediated extension reaction.

“Recipient Nucleic Acid Molecule”: The term “recipient nucleic acid molecule”, as used herein, refers to a nucleic acid molecule that may be joined with one or more variant nucleic acid molecules containing one or more targeted positions or regions of diversity. In certain embodiments, a recipient nucleic acid molecule is a vector or portion of a vector. In certain embodiments, joining a recipient nucleic acid molecule with one or more variant nucleic acid molecules generates a circular nucleic acid molecule. In certain embodiments, more than one recipient nucleic acid molecule is joined with one or more variant nucleic acid molecules. One of ordinary skill in the art will be able to select appropriate and/or desirable recipient nucleic acid molecules for use with systems of the present invention.

“Saturation mutagenesis”: The term “saturation mutagenesis” as used herein refers to a method of generating a comprehensive set of alterations at one or more positions in a given nucleic acid or polypeptide sequence such that all possible nucleotide or amino acid residues are represented. For example, when using only naturally occurring nucleotide residues, saturation mutagenesis of a single position in a nucleic acid sequence will generally result in four variant nucleic acid molecules that collectively comprise the bases adenine, cytosine, guanine and thymine/uracil (depending on whether the nucleic acid sequence comprises deoxyribonucleotides or ribonucleotides) at the mutagenized nucleotide position. In some embodiments, nucleotides that do not occur naturally may be included in the saturation mutagenesis, resulting in a plurality of variant nucleic acid molecules that collectively comprise the non-naturally occurring nucleotides in addition to the four naturally occurring nucleotides at the mutagenized position. “Saturation mutagenesis” of a single position in an amino acid sequence results in twenty different polypeptides (or more if non-natural amino acids are employed). It will be understood that saturation mutagenesis of a given position in a polypeptide may be accomplished through production of a plurality of variant nucleic acid molecules encoding every possible amino acid at the selected position. It will be further understood that more than one position in a particular nucleic acid or polypeptide sequence may be subjected to saturation mutagenesis, for example by introducing diversity into one or more targeted positions or regions of the nucleic acid of polypeptide sequence using systems of the present invention.

“Substantially similar”: As used herein, the term “substantially similar”, as applied to nucleic acid sequences, refers to two or more nucleic acid molecules or portions of nucleic acid molecules, which nucleic acid molecules or portions contain one or more identical nucleotides positioned at corresponding positions along the nucleic acid molecule or portion. In certain embodiments, the term substantially similar refers to nucleic acid molecules or portions of nucleic acid molecules whose nucleotide sequences are, for example, 50, 55, 60, 65, 70, 75, 80, 85 or 90 percent identical over a given length of the nucleic acid molecule or portion. In certain embodiments, the term substantially similar refers to nucleic acid molecules or portions of nucleic acid molecules whose nucleotide sequences are, for example, 91, 92, 93, 94, 95, 96, 97, 98, or 99 percent identical over a given length of the nucleic acid molecule or portion. The length of the nucleic acid molecule or portion over which two or more nucleic acid molecules or portions are substantially similar may be, for example, 10, 15, 20, 25, 30, 35, 40, 45, 50, 60, 70, 80, 90, 100, 200, 300, 400, 500, 600, 700, 800, 900, 1000 or more nucleotides. In some embodiments, two nucleic acid molecules or portions of nucleic acid molecules are substantially similar if they are able to hybridize to the same portion of another nucleic acid molecule under stringent hybridization conditions. Two nucleic acid molecules may be substantially similar even though one nucleic acid molecule contains one or more nucleotide residues that do not correspond in position to any residue present in the other (e.g., one nucleic acid molecule may contain one or more insertions, deletion and/or rearrangements as compared to another nucleic acid molecule that is otherwise substantially similar). As will be clear from the context, the term “substantially similar”, as applied to polypeptide sequences, alternately refers to two or more polypeptides, which polypeptides contain one or more identical or similar amino acids at corresponding positions along the polypeptide. In some embodiments, amino acids are similar to each other if their side chains are structurally similar. For example, amino acids with aliphatic side chains, including glycine, alanine, valine, leucine, and isoleucine, are similar; amino acids having aliphatic-hydroxyl side chains, including serine and threonine, are similar; amino acids having amide-containing side chains, including asparagine and glutamine, are similar; amino acids having aromatic side chains, including phenylalanine, tyrosine, and tryptophan, are similar; amino acids having basic side chains, including lysine, arginine, and histidine, are similar; and amino acids having sulfur-containing side chains, including cysteine and methionine, are similar. In some embodiments, amino acids are similar to each other if their side chains exhibit similar chemical properties. For example, in certain embodiments, amino acids that comprise hydrophobic side chains are similar. In some embodiments, amino acids are similar if their side chains are of similar molecular weight or bulk. For example, amino acids may be similar if their side chains exhibit a minimum/maximum molecular weight or take up a minimum/maximum amount of space. In certain embodiments, the term substantially similar refers to polypeptides or portions of polypeptides whose amino acid sequences are, for example, 50, 55, 60, 65, 70, 75, 80, 85 or 90 percent identical or similar over a given length of the polypeptide or portion. In certain embodiments, the term substantially similar refers to polypeptides or portions of nucleic acid molecules whose amino acid sequences are, for example, 91, 92, 93, 94, 95, 96, 97, 98, or 99 percent identical or similar over a given length of the polypeptide or portion. The length of the polypeptide or portion over which two or more polypeptides or portions are substantially similar may be, for example, 5, 6, 7, 8, 9, 10, 15, 20, 25, 30, 35, 40, 45, 50, 60, 70, 80, 90, 100, 200, 300, 400, 500, 600, 700, 800, 900, 1000 or more amino acids.

“Terminator”: As will be clear from the context, the term “terminator” as used herein refers to either a terminator nucleotide or a terminator structure (see definitions of “Terminator Nucleotide” and “Terminator Structure”, infra), which terminator nucleotide or terminator structure is not capable of being copied by at least one polymerase in a polymerase-mediated extension reaction under at least one set of polymerization conditions.

“Terminator Nucleotide”, “Terminator Residue”: The terms “terminator nucleotide” and “terminator residue” as used herein refer to a nucleotide or nucleotide analog that is not capable of being copied by at least one polymerase in a polymerase-mediated extension reaction under at least one set of polymerization conditions. A given terminator nucleotide may be capable of being copied by a different polymerase under otherwise identical or similar conditions. Additionally or alternatively, a given terminator nucleotide may be capable of being copied by the same polymerase under a different set of polymerization conditions. In certain embodiments, the terminator nucleotide is contained in a primer that is used in a polymerase-mediated extension reaction. Furthermore, a primer containing a terminator nucleotide may be used in conjunction with any method disclosed in U.S. Pat. No. 6,358,712, U.S. patent application Ser. No. 10/272,531 and/or in U.S. patent application Ser. No. 10/383,135, each of which is incorporated herein by reference in its entirety. As but one non-limiting example, a primer may contain one or more ribonucleotide residues that are not copied by at least one polymerase used in the polymerase-mediated extension reaction. As another non-limiting example, a primer may contain one or more 2′-O-methyl residues that are not copied by at least one polymerase used in the polymerase-mediated extension reaction.

“Terminator Structure”: The term “terminator structure” as used herein refers to a structural feature of nucleic acid molecule, at a position in relation to the phosphate backbone where a nucleotide is normally located, that does not permit one or more polymerases used in a polymerization reaction to continue polymerization beyond that structural feature and copy nucleotides beyond the structure feature under at least one set of polymerization conditions. Any physical moiety that functions to stop the polymerase from copying nucleotides beyond a given position along the nucleic acid strand is a terminator structure. Additionally, the absence of a nucleotide residue at a given position along the phosphate backbone (i.e., an “abasic site”) may be a terminator structure if it functions to stop the polymerase from copying nucleotides beyond that abasic site. In certain embodiments, a polymerase that is not able to continue polymerization beyond a terminator structure may be capable of continuing polymerization beyond the terminator structure under one or more different sets of polymerization conditions. Additionally or alternatively, one or more different polymerases may be capable of continuing polymerization beyond a terminator structure under the same or a similar set of polymerization conditions. In certain embodiments, a terminator structure is contained in a primer. As used herein, a structural feature of a terminator structure does not refer to a nucleotide or a nucleotide analog (see definition of “Terminator Nucleotide”, supra).

“Variant Nucleic Acid Molecule”: The term “variant nucleic acid molecule”, as used herein, refers to a member of a plurality of nucleic acid molecules that contain one or more positions or regions of diversity. In certain embodiments, a plurality of variant nucleic acid molecules is generated by subjecting a template nucleic acid molecule to a polymerase-mediated extension reaction using one or more degenerate primer sets. The diversity present in a plurality of generated variant nucleic acid molecules will reflect the diversity present in the degenerate primer set.

### DETAILED DESCRIPTION OF CERTAIN EMBODIMENTS

The present invention provides novel systems that may be used to introduce diversity into one or more targeted positions or regions of a nucleic acid molecule, for example by generating a plurality of variant nucleic acid molecules that contain one or more targeted positions or regions of diversity. In certain embodiments, variant nucleic acid molecules of the plurality encode polypeptides.

Additional and alternative embodiments of the invention are discussed in detail below. Those of ordinary skill in the art will understand, however, that various modifications to these embodiments are within the scope of the appended claims. It is the claims and equivalents thereof that define the scope of the present invention, which is not and should not be limited to or by this description of certain embodiments.

Template Nucleic Acid Molecules

Any nucleic acid molecule that is susceptible to polymerase-mediated extension may be used as a template nucleic acid molecule in accordance with methods and systems disclosed herein. In certain embodiments, a template nucleic acid molecule is a naturally occurring molecule. For example, a template nucleic acid molecule may be isolated from the genome of a particular organism or cell. Numerous methods of isolating nucleic acids from organisms or cells are known to those of ordinary skill in the art. In certain embodiments, nucleic acids isolated from the genomes of organisms or cells may be used directly as template nucleic acid molecules. Alternatively, nucleic acids isolated from the genomes of organisms or cells may be amplified or otherwise replicated before being used as template nucleic acid molecules, for example, by PCR. Additionally or alternatively, nucleic acid fragments isolated from the genomes of organisms or cells may be cloned into a vector, wherein the vector containing the nucleic acid fragment is used as a template nucleic acid molecule. Non-limiting examples of vectors include plasmid vector, yeast artificial chromosomes (“YACs”), bacterial artificial chromosomes (“BACs”) and phage vectors, including, but not-limited to phagemids, cosmids, PAC vectors and bacteriophages. Those of ordinary skill in the art will be aware of other suitable vectors that may be used to clone genomic nucleic acid fragments. In certain embodiments, a vector lacking any cloned nucleic acid sequence is used as a template nucleic acid molecule.

In certain embodiments, a nucleic acid molecule or fragment isolated from the genome of an organism or cell is used as a template nucleic acid molecule. In certain embodiments, a template nucleic acid molecule comprises a fragment of a genome. In certain embodiments, a template nucleic acid molecule comprises a fragment of a chromosome. In certain embodiments, a template nucleic acid molecule comprises a fragment of a gene. For example, genomic DNA of most eukaryotic genes includes regions that code for genes (“exons”) as well as regions between the exons that do not code for genes (“introns”). Introns are generally spliced out upon the transcription of genomic DNA into RNA, leaving only exons in the mature transcript. Sequences within the intron itself are often critical in determining whether and how efficiently the intron will be spliced out. Thus, by introducing diversity into targeted positions or regions of the spliced introns, systems of the present invention provide a useful tool for studying the nucleotide sequences that are important for proper splicing. In certain embodiments, a template nucleic acid molecule comprises a fragment of a functional domain of a gene.

Alternatively, a non-genomic nucleic acid molecule may be isolated and used as a template nucleic acid molecule. For example, a template nucleic acid molecule may be derived from the nucleotide sequence of one or more mRNAs expressed in a particular organism or cell. In certain embodiments, mRNA is isolated and reverse-transcribed to generate one or more cDNAs, which cDNAs are used as template nucleic acid molecules. In certain embodiments, systems of the present invention are used to introduce targeted diversity into one or more positions or regions of cDNAs that encode a polypeptide. In certain embodiments, systems of the present invention are used to introduce targeted diversity into one or more positions or regions of cDNAs that do not encode a polypeptide.

In certain embodiments, a template nucleic acid molecule is expressed mRNA itself. For example, mRNA may be isolated from a cell or organism of interest and subjected to polymerase-mediated extension using a polymerase that is capable of using ribonucleotide residues as templates. In certain embodiments, a polymerase used in a polymerase-mediated extension reaction is capable of using both ribonucleotide residues and deoxyribonucleotide residues as templates under one or more polymerization conditions.

In certain embodiments, a template nucleic acid molecule is a molecule that does not occur naturally. For example, a template nucleic acid molecule may comprise two or more naturally occurring sequences, which sequences are not naturally present in the same nucleic acid molecule. Additionally or alternatively, a template nucleic acid molecule may comprise two or more naturally occurring sequences, which sequences are present in the same nucleic acid molecule but are present in a positional relationship different from that in which they occur in the template nucleic acid molecule. Additionally or alternatively, a template nucleic acid molecule may comprise portions of two or more homologous nucleic acid sequences. In certain embodiments, a template nucleic acid molecule comprises a nucleic acid sequence that encodes a polypeptide in which one or more regions or domains of the polypeptide have been replaced with one or more homologous regions or domains from a homologous polypeptide. In certain embodiments, a template nucleic acid molecule comprises a nucleic acid sequence that encodes a polypeptide into which a nucleic acid sequence that encodes an exogenous region or domain has been inserted, which exogenous region or domain is not naturally found in that polypeptide. In certain embodiments, the exogenous region or domain is not naturally found in a homolog of the polypeptide into which the exogenous region or domain is inserted.

Additionally or alternatively, a template nucleic acid molecule may comprise one or more artificial nucleic acid sequences that do not occur naturally in any organism. In certain embodiments, a template nucleic acid molecule may comprise both naturally and non-naturally occurring nucleic acid sequences. For example, a template nucleic acid molecule may comprise a naturally occurring nucleic acid sequence which has been mutagenized, either in vivo or in vitro, at one or more positions or regions, such that the mutagenized nucleic acid sequence is not found in nature. Such mutagenized nucleic acid sequences may possess unexpected and/or enhanced desirable characteristics. Systems of the present invention may be advantageously used to introduce targeted regions of diversity into one or more positions or regions of such mutagenized nucleic acid sequences in order to further enhance the desirable characteristics. In certain embodiments, a template nucleic acid molecule is completely artificial. A completely artificial template nucleic acid sequence may be synthesized by any of a variety of techniques well known to those of ordinary skill in the art. In certain embodiments, a template nucleic acid molecule is generated by the hand of man.

In certain embodiments, a template nucleic acid molecule is created by any of the methods described in U.S. patent application Ser. No. 11/271,561, incorporated herein by reference in its entirety. For example, a template nucleic acid molecule may be generated by building a ladder complex of partially complementary oligonucleotides. A ladder complex generated according to any of the methods described in the Ser. No. 11/271,561 application may be converted into a double-stranded or partially double-stranded template nucleic acid molecule prior to introducing diversity into one or more targeted positions or regions using any of the systems described herein. Alternatively, a ladder complex generated according to any of the methods described in the Ser. No. 11/271,561 application may be converted into a double-stranded or partially double-stranded product molecule simultaneous to introducing diversity into one or more targeted positions or regions using any of the systems described herein.

In certain embodiments, one or more variant nucleic acid molecules into which targeted diversity is introduced according to systems of the present invention may themselves be used as template nucleic acid molecules. Such iterative diversity generating methods permit sequential and/or simultaneous introduction of targeted diversity into one or more positions or regions of an original nucleic acid molecule.

Introducing Sequence Diversity into a Nucleic Acid Molecule

Systems of the present invention are useful for introducing diversity into one or more targeted positions or regions of a nucleic acid molecule. In certain embodiments, diversity is introduced into a targeted position or region of a template nucleic acid molecule by providing two primers and a template nucleic acid molecule, wherein the primers are extended in opposite directions against the template nucleic acid molecule in a polymerase-mediated extension reaction. In certain embodiments, a generated nucleic acid molecule into which diversity has been introduced includes terminal single-stranded overhangs, which single-stranded overhangs are capable of annealing to each other. In certain embodiments, the annealed single-stranded overhangs are subjected to an optional ligation step prior to further manipulation and/or utilization of the generated nucleic acid molecule.

In certain embodiments, diversity is introduced into a targeted position or region of a template nucleic acid by employing as least one degenerate primer set in a polymerase-mediated extension reaction, which degenerate primer set contains one or more positions or regions of degeneracy. Extending a degenerate primer set against a template nucleic acid molecule in a polymerase-mediated extension reaction results in a plurality of degenerate nucleic acid molecules whose degeneracy reflects the degeneracy present in the degenerate primer set.

In certain embodiments, a degenerate position or region in a primer is completely degenerate such that all possible alternative nucleotides are represented. For example, in the context of naturally occurring nucleotide residues, a degenerate position is completely degenerate where the degenerate position is represented by nucleotides containing the nucleotide bases adenine, cytosine, guanine and thymidine. In certain embodiments, it may be desirable to provide a primer that is completely degenerate at one or more positions. For example, a primer containing one or more completely degenerate positions may be used to generate a plurality of variant nucleic acid molecules that contains maximal sequence diversity at the degenerate position(s). Primers containing one or more completely degenerate positions are useful for the introduction of diversity into a targeted position or region of a nucleic acid molecule to achieve saturation mutagenesis of one or more nucleotide positions.

In certain embodiments, a degenerate position or region in a primer is less than completely degenerate. For example, a degenerate position is less than completely degenerate where the degenerate position is represented by three residues that contain nucleotide bases selected from the group consisting of adenine, cytosine, guanine and thymidine. In certain embodiments, a less than completely degenerate position is represented by two nucleotide residues. In certain embodiments, two nucleotide residues used to represent a less than completely degenerate position are purines, e.g., adenine and guanine. In certain embodiments, two nucleotides used to represent a less than completely degenerate position are pyrimidines, e.g., cytosine and thymidine. In certain embodiments, two nucleotides used to represent a less than completely degenerate position are capable of base pairing with each other, e.g., adenine and thymidine, or guanine and cytosine.

In certain embodiments, a degenerate position is represented by a nucleotide base that does not occur naturally. In certain embodiments, a polymerase used in a polymerase-mediated extension reaction is able to use a nucleotide that does not occur naturally as a template. For example, a polymerase may use a non-naturally occurring base as a template to incorporate another non-naturally occurring base into the extension product. Additionally or alternatively, a polymerase may use a non-naturally occurring base as a template to incorporate a naturally occurring base into the extension product. Additionally or alternatively, a polymerase may use a naturally occurring base as a template to incorporate a non-naturally occurring base into the extension product. In certain embodiments, a degenerate position is represented both by a nucleotide base that is does not occur naturally and by a nucleotide base that does occur naturally.

A single primer may contain two or more degenerate positions. Such degenerate positions may independently be completely or less than completely degenerate. In certain embodiments, two or more degenerate positions are positioned adjacent to each other along the length of the primer. In certain embodiments, two or more degenerate positions are separated by one or more non-degenerate positions.

Systems of the present invention are useful for introducing diversity into a targeted position or region of a circular nucleic acid molecule. For example, two degenerate primer sets may be designed that anneal to the circular template nucleic acid molecule and that are extended in opposite directions in a polymerase-mediated extension reaction, resulting in a plurality of linear degenerate product molecules. Members of such a plurality of linear product molecules are then re-circularized, resulting in a plurality of degenerate circular nucleic acid molecules, the degeneracy of the plurality reflecting the degeneracy present in the degenerate primer set. In certain embodiments, re-circularization is achieved via self-ligation of the plurality of degenerate linear product molecules.

In certain embodiments, re-circularization is achieved by designing primers that result in terminal single-stranded overhangs on the plurality of degenerate linear product molecules, which terminal single-stranded overhangs are capable of annealing to each other. In certain embodiments, terminal single-stranded overhangs are generated through the use of terminator nucleotides and/or terminator structures present in the primers. In certain embodiments, terminal single-stranded overhangs are generated by manipulating the product of the polymerase-mediated extension reaction, for example, by removing terminal nucleotides from one strand of the polymerase-mediated extension product. In certain embodiments, the removed nucleotides comprise ribonucleotides that are removed by exposure to high pH, certain nucleases that remove ribonucleotides from a double-stranded RNA-DNA hybrid molecule, or by any of a number of techniques and methods known to those of ordinary skill in the art.

By incubating such a plurality of linear degenerate product molecules containing single-stranded overhangs under conditions that permit annealing of the overhangs, the plurality of degenerate linear product molecules is thereby converted to a plurality of degenerate circular product molecules. In certain embodiments, the annealed plurality of degenerate circular product molecules may be subjected to a ligation reaction, either in vivo or in vitro.

In certain embodiments, a degenerate primer set may be designed such that a polymerase-mediated extension reaction produces a plurality of variant nucleic acid molecules, members of which contain two terminal single-stranded overhangs, which overhangs are not perfectly complementary. In certain embodiments, even though the terminal single-stranded overhangs are not perfectly complementary, such overhangs are nevertheless sufficiently complementary over a sufficient length to anneal to each other under at least one set of annealing conditions. Upon annealing, the terminal single-stranded overhangs form a double-stranded region that contains one or more mismatched base pairs that do not form hydrogen bonds in the ordinary Watson-Crick sense at the position(s) where the overhangs are not perfectly complementary.

In certain embodiments, a plurality of variant nucleic acid molecules is generated by subjecting a template nucleic acid molecule to polymerase-mediated extension using one degenerate primer set and one non-degenerate primer. In certain embodiments, the non-degenerate primer comprises a sequence identical to the template nucleic acid molecule. In certain embodiments, the non-degenerate primer comprises a sequence that is not identical to a template nucleic acid molecule. For example, the non-degenerate primer may comprise a sequence that differs from a template nucleic acid molecule at one or more positions. Upon annealing, such terminal single-stranded overhangs form a double-stranded region that contains one or more mismatched base pairs at positions where the sequences of the degenerate primer set and the non-degenerate primer are not complementary.

In certain embodiments, mismatched base pairs are resolved in vitro, for example through the use of an in vitro mismatch repair system or kit. In certain embodiments, mismatched base pairs are resolved in vivo, for example by transforming the annealed product molecule into a cell such as E. coli, which is known to repair mismatched base pairs. One of ordinary skill in the art will be aware of other appropriate and useful in vitro or in vivo systems that may be used to resolve mismatched base pairs.

In certain embodiments, use of an in vitro or in vivo repair system reduces the number of primers required to be present in a degenerate primer set to achieve a desired level of degeneracy in one or more targeted regions of a nucleic acid molecule. For example, a single nucleic acid residue may be subjected to saturation mutagenesis by providing one non-degenerate primer, which comprises a sequence identical to that of a template nucleic acid molecule, and a degenerate primer set, which comprises one or more positions of degeneracy represented by the three alternate nucleotide residues that are not present at the corresponding positions in a template nucleic acid molecule. The primers are designed to generate single stranded overhangs upon polymerase-mediated extension (or treatment subsequent to polymerase-mediated extension), which overhangs comprise the degenerate position and which overhangs are sufficiently complementary over a sufficient length to anneal to each other. Upon polymerase-mediated extension, a plurality of three product molecules will be generated. Each of the three product molecules will comprise the sequence of the non-degenerate primer in one overhang. The other overhang will comprise one of three sequences containing one of the three nucleotides represented by the degenerate position. The overhangs are then annealed and the mismatched base pairs are resolved. Mismatch resolution results in four possible product molecules comprising each of the four possible nucleotides at the degenerate position. In certain embodiments, an in vitro or in vivo repair system may be used to resolve mismatched base pairs created at more than one degenerate position. For example, mismatches may be resolved by introducing a generated plurality of product molecules into a host organism that is capable of resolving mismatches. As one non-limiting example, such a generated plurality may be introduced into E. coli. In certain embodiments, a generated plurality of product molecules is introduced into a host organism that favors one strand over the other for use as a template in in vivo repair. Therefore, it may be possible to take advantage of such preferential mismatch repair in order to direct and/or control the mismatch repair products, and/or to minimize the number of primers required to achieve a desired level of degeneracy.

In certain embodiments, a template nucleic acid molecule is subjected to two or more iterative polymerase-mediated extension reactions. In some embodiments, iterative polymerase-mediated extension reactions may be used to generate diversity in a single position or region of a nucleic acid molecule. For example, a first plurality of variant nucleic acid molecules that contains diversity within a single targeted position or region may be generated. One or more members of such a first plurality may then be used as template nucleic acid molecules in one or more subsequent polymerase-mediated extension reactions to generate a subsequent plurality of variant nucleic acid molecules that contains further diversity in the originally targeted position or region.

In certain embodiments, a first plurality of generated variant nucleic acid molecules is introduced into a host cell for amplification and/or propagation prior to introducing further diversity in subsequent polymerase-mediated extension reactions. For example, such a first plurality may be introduced into E. coli, and one or more members of the plurality may be recovered for subsequent polymerase-mediated extension reactions. In certain embodiments, a single member of such a first plurality is recovered for use as a template nucleic acid molecule in subsequent rounds of polymerase-mediated extension. In certain embodiments, multiple members of such a first plurality are recovered for use as a template nucleic acid molecules in subsequent rounds of polymerase-mediated extension.

In certain embodiments, a first plurality of generated variant nucleic acid molecules is subjected to subsequent rounds of polymerase-mediated extension without introducing the first plurality into a host cell. In certain embodiments, members of a first generated plurality are subjected to ligation prior to subsequent rounds of polymerase-mediated extension. In certain embodiments, members of a first generated plurality are not subjected to ligation prior to subsequent rounds of polymerase-mediated extension. For example, one or more members of a first generated plurality of nucleic acid molecules may comprise single-stranded termini that are at least partially complementary. Such termini may be able to anneal such that the annealed nucleic acid molecule is a desirable substrate for subsequent rounds of polymerase-mediated extension. In certain embodiments, a polymerase is used in subsequent polymerase-mediated extension reaction that is capable of utilizing terminator nucleotides as templates and/or reading through such terminator nucleotides at some frequency. For example, such embodiments are useful where diversity is introduced into a template nucleic acid molecule in a first region using terminator nucleotides, and further diversity is subsequently introduced into a second region in one or more additional polymerase-mediated extension reactions, which additional reactions use terminator nucleotides present in the first region as templates. One of ordinary skill in the art will be aware of appropriate and useful polymerases, polymerization conditions, terminator nucleotides and other reaction components and/or conditions that permit such embodiments.

Additionally or alternatively, iterative polymerase-mediated extension reactions may be used to create diversity in two or more positions or regions of a template nucleic acid molecule. For example, a first plurality of variant nucleic acid molecules that contains diversity within a single targeted position or region may be generated. One or more members of such a first plurality may then be used as template nucleic acid molecules in one or more subsequent polymerase-mediated extension reactions to generate a subsequent plurality of variant nucleic acid molecules that contain further diversity in one or more additional targeted positions or regions.

Introducing targeted diversity via iterative polymerase-mediated extension reactions may be useful where a large amount of diversity is to be introduced and it is desirable to introduce smaller amounts of diversity in a step-wise fashion to facilitate screening, to reduce the number of primers required in any given extension reaction, and/or for any other logistical or experimental constraint the practitioner deems important. Additionally or alternatively, iterative polymerase-mediated extension reactions may be useful where it is desirable to screen for or identify one or more characteristics of the first plurality of variant nucleic acid molecules before introducing further diversity. For example, a first plurality of generated variant nucleic acid molecules may be tested or screened prior to the subsequent polymerase-mediated extension reaction(s) and one or more members of the first plurality with one or more desirable characteristics may be selected for use as template nucleic acid molecules in subsequent polymerase-mediated extension reaction(s). This process may be repeated to generate further diversity in one or more targeted positions or regions. In certain embodiments, members of the first plurality of variant nucleic acid molecules are used as template nucleic acid molecules in one or more subsequent rounds of polymerase-mediated extension without isolation or screening.

In certain embodiments, a template nucleic acid molecule is circular and the generated plurality of variant nucleic acid molecules are re-circularized after polymerase-mediated extension. For example, a polymerase-mediated extension reaction may be designed to generate a plurality of variant nucleic acid molecules that include terminal single-stranded regions that are capable of annealing to each other. Alternatively, a polymerase-mediated extension may be designed to generate a plurality of variant nucleic acid molecules that may be subjected to further manipulation to generate terminal single-stranded regions that are capable of annealing to each other. In certain embodiments, such an annealed plurality of variant nucleic acid molecules is subjected to a ligation reaction and/or propagated in a host cell. A generated plurality of variant nucleic acid molecules may be tested or screened and a subset of variants with one or more desirable properties may be selected for use as templates in one or more subsequent rounds of polymerase-mediated extension, in which a new plurality of variant nucleic acid molecules that contain further diversity in the originally targeted position or region are created.

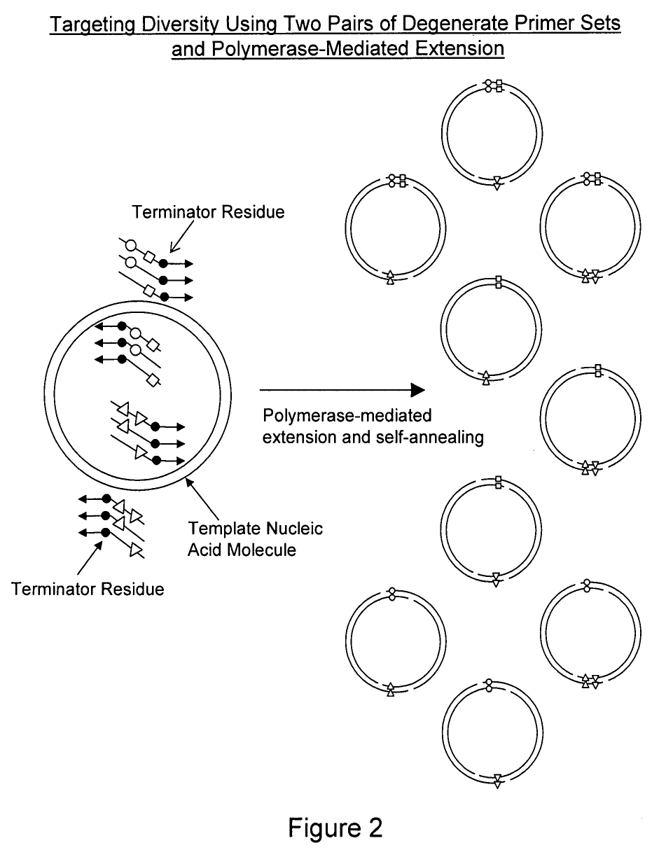

In certain embodiments, systems of the present invention are used to introduce diversity into two or more targeted positions or regions of a nucleic acid molecule by subjecting a template nucleic acid molecule to two or more polymerase-mediated extension reactions (for example, see the embodiment shown in FIG. 2). In certain embodiments, each polymerase-mediated extension reaction generates a plurality of variant nucleic acid molecules. In certain embodiments, each plurality corresponds to a different region of a template nucleic acid molecule, and members of each plurality can be combined with members of at least one other plurality to reconstitute one or more variant nucleic acid molecules that are substantially similar to the template nucleic acid molecule and that contain one or more targeted regions or positions of diversity.

To give but one example, targeted diversity may be introduced into two positions or regions of a template nucleic acid molecule by subjecting a template nucleic acid molecule to two polymerase-mediated extension reactions using two pairs of degenerate primer sets. A first set of degenerate primers is used to generate a first plurality of variant nucleic acid molecules, each of which contains a first and second overhang (or may be treated to generate a first and second overhang), which first and second overhangs do not anneal to each other under at least one set of conditions. A second set of degenerate primers is used to generate a second plurality of variant nucleic acid molecules, each of which contains a third and fourth overhang (or may be treated to generate a third and fourth overhang), which third overhang anneals to the first overhang of the first plurality of variant nucleic acid molecules and which fourth overhang anneals to the second overhang of the first plurality of variant nucleic acid molecules, but which third and fourth overhangs do not anneal to each other under at least one set of conditions. The first and second pluralities of generated variant nucleic acid molecules may be annealed to form a third plurality of variant nucleic acid molecules. By choosing primer pairs that introduce diversity into the positions or regions targeted by the first and second (and third and fourth) overhangs and annealing the first and second generated plurality of variant nucleic acid molecules to form a third plurality of variant nucleic acid molecules, diversity may be introduced into two positions or regions of a template nucleic acid molecule simultaneously upon joining members of the first and second plurality of variant nucleic acid molecules. The third plurality of variant nucleic acid molecules may be used as a template in which further diversity is subsequently introduced into the same or different positions or regions.

In certain embodiments, the two or more polymerase-mediated extension reactions are performed simultaneously. In certain embodiments, the two or more polymerase-mediated extension reactions are performed sequentially.

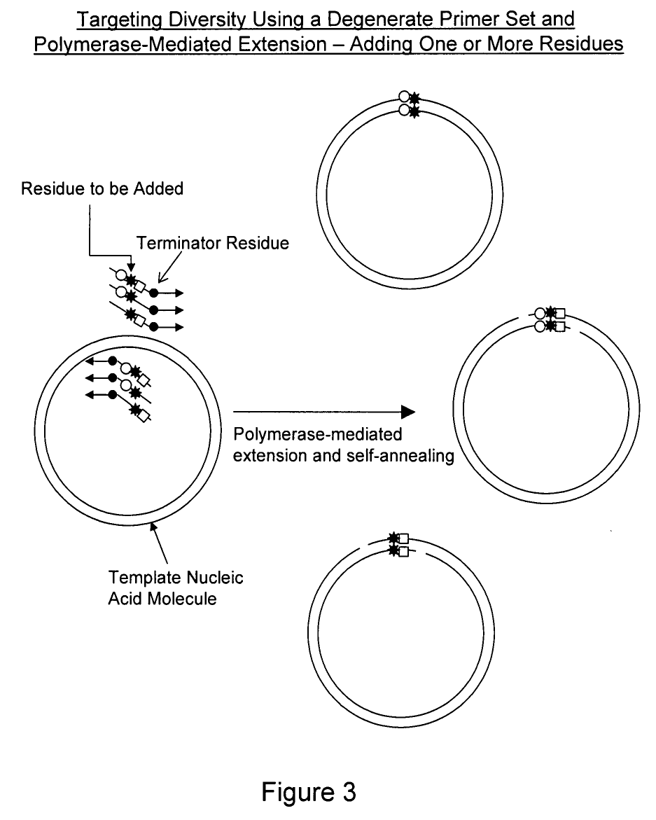

In certain embodiments, systems of the present invention are used to introduce one or more nucleotide residues that do not correspond in position to any nucleotide residue of a template nucleic acid molecule. For example, a template nucleic acid molecule may be subjected to a polymerase-mediated extension reaction using one or more degenerate primer sets that are substantially similar over their lengths to the template nucleic acid molecule, but that contain one or more nucleotides to be introduced that do not correspond in position to any nucleotide of the template nucleic acid molecule (for example, see the embodiment shown in FIG. 3). In certain embodiments, a nucleotide position to be introduced is represented by a single nucleotide residue. In such embodiments, variant nucleic acid molecules into which one or more positions or regions of diversity have been targeted will contain the additional nucleotide, which extra nucleotide is not degenerate. In certain embodiments, a nucleotide position to be introduced is degenerate in that it is represented by more than one nucleotide residue. In such embodiments, the variant nucleic acid molecules into which one or more positions or regions of diversity have been targeted will contain the additional nucleotide, which extra nucleotide is degenerate. In certain embodiments, both degenerate and non-degenerate nucleotides that do not correspond in position to any nucleotide present in a template nucleic acid molecule may be introduced in one or more polymerase-mediated extension reactions.

In certain embodiments, systems of the present invention are used to introduce multiple nucleotide residues, none of which correspond in position to any nucleotide residue of a template nucleic acid molecule. As but one non-limiting example, a set of three nucleotide residues may be introduced, either sequentially or simultaneously, into a nucleic acid molecule that encodes a polypeptide, wherein such a set of three encodes a desired amino acid. By introducing such a set of three nucleic acid residues that encodes a particular amino acid in frame with the polypeptide-encoding open reading frame, a nucleic acid molecule may be engineered to express a polypeptide that contains an introduced amino acid at a desired position in the peptide sequence. As will be clear to those of ordinary skill in the art, multiple sets of three nucleotide residues, wherein each set of three encodes a desired amino acid, may be introduced to generate a nucleic acid that encodes an engineered polypeptide that contains multiple introduced amino acids.

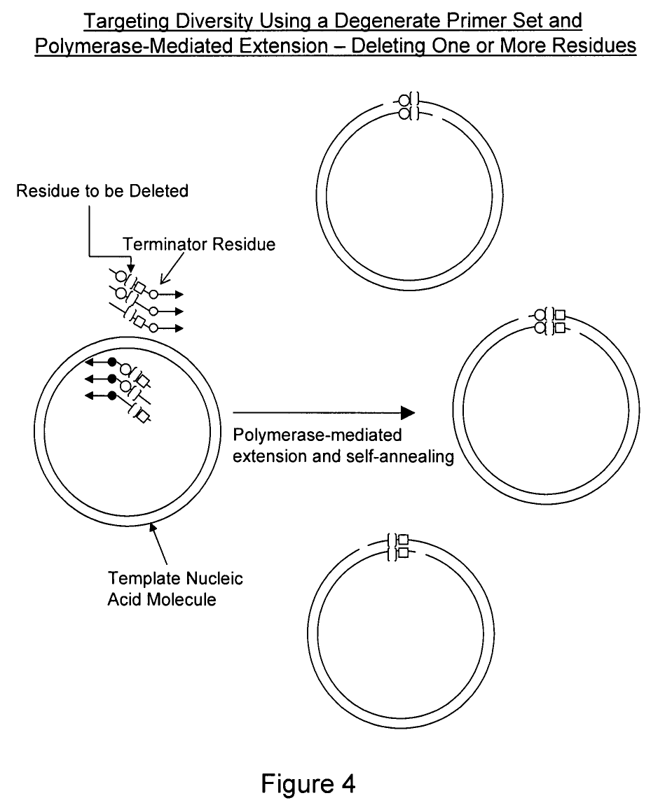

In certain embodiments, systems of the present invention are used to delete one or more nucleotide residues present in a template nucleic acid molecule. For example, a template nucleic acid molecule may be subjected to a polymerase-mediated extension reaction using one or more degenerate primer sets that are substantially similar over their lengths to the template nucleic acid molecule, but that lack one or more nucleotides as compared to the template nucleic acid molecule (for example, see the embodiment shown in FIG. 4). In such embodiments, a template nucleic acid molecule contains one or more nucleotides that do not correspond in position to any nucleotide of the variant nucleic acid molecule(s).

In certain embodiments, systems of the present invention are used to generate variant nucleic acid molecules that both contain one or more nucleotide residues that do not correspond in position to any nucleotide present in a template nucleic acid molecule and that lack one or more nucleotide residues present in a template nucleic acid molecule.

In certain embodiments, systems of the present invention are used to generate variant nucleic acid molecules that contain one or more altered, non-degenerate nucleotide positions that correspond in position to a nucleotide residue of a template nucleic acid molecule, but that differ in identity from the corresponding nucleotide residue of the template nucleic acid molecule. Such embodiments may be useful when it is desired to introduce a non-variant nucleotide substitution into a nucleic acid molecule. For example, if a template nucleic acid molecule contains a guanosine residue at a particular position, primers may be designed such that generated variant nucleic acid molecules contain a different residue at that position. In certain embodiments, a variant nucleic acid molecule encodes a polypeptide and the altered, non-degenerate nucleotide residue that is introduced alters the encoded polypeptide sequence. In certain embodiments, the altered nucleotide residue is a naturally occurring residue. In certain embodiments, the altered nucleotide residue is a non-naturally occurring residue. In certain embodiments, systems of the present invention are used to introduce both altered, non-degenerate nucleotide residues as well as degenerate positions represented by more than one nucleotide residue into variant nucleic acid molecules. Additionally or alternatively, generated variant nucleic acid molecules may contain one or more nucleotide residues that do not correspond in position to any nucleotide present in a template nucleic acid molecule and/or may lack one or more nucleotide residues present in the template nucleic acid molecule.

Systems of the present invention may be used to create one or more libraries of nucleic acid molecules in which phylogenetic analysis has been used to guide the design of a degenerate primer set such that the diversity targeted to one or more positions or regions of a template nucleic acid molecule is restricted to only particular nucleotide positions and/or only a subset of the possible nucleotides are encoded at those positions. Homologous nucleic acid sequences may differ in their ability to regulate a nearby gene, their ability to promote translation of a messenger RNA, their ability to regulate stability of a messenger or other RNA or any other property or characteristic. These homologous nucleic acid sequences may be very similar or identical over a given portion of their lengths, but may be quite divergent at one or more positions or regions, indicating that these positions or regions may play a role in the different properties or characteristics of the nucleic acid sequences. Systems of the present invention may be advantageously used to introduce additional diversity into the divergent positions or regions of one or more of the homologous nucleic acid sequences. Additionally or alternatively, systems of the present invention may be advantageously used to introduce additional diversity into the non-divergent positions or regions of one or more of the homologous nucleic acid sequences. In certain embodiments, divergent and/or non-divergent positions or regions of the homologous nucleic acid sequences are subject to saturation mutagenesis. In certain embodiments, divergent and/or non-divergent positions or regions of the homologous polypeptides are subject to mutagenesis that does not reach saturation.

Introducing Diversity into a Polypeptide-Encoding Nucleic Acid Molecule

Polypeptides have become increasingly important therapeutic, agricultural and commercial agents. More and more discovery and research is directed towards the identification of polypeptides that function as useful agents or that are themselves targets of a drug molecule. In certain instances, a change in the amino acid sequence of a polypeptide of interest can alter its properties. For example, altering the amino acid sequence of the active site of an enzyme can shift the substrate specificity of the enzyme, the rate of catalysis, and/or any other property of the enzyme. Similarly, altering the amino acid sequence of a polypeptide that binds one or more particular ligands may alter the ligand-binding specificity of the polypeptide and/or alter the strength with which the polypeptide binds the ligand(s). Identifying such amino acid sequences and optimizing them to generate a polypeptide with one or more desired properties is a critical challenge in the development of new and useful variants of known polypeptide sequences. With the advent of large-scale sequencing projects, an overwhelming number of genes predicted to encode one or more polypeptides are now known. Even with such extensive knowledge of predicted polypeptide sequence data, researchers are often hampered in their efforts to develop therapeutically, agriculturally or commercially useful variants of these polypeptides by a lack of functional knowledge about these predicted polypeptides and their domains.

In certain embodiments, systems of the present invention are useful for the introduction of diversity into a targeted position or region of a nucleic acid molecule that encodes a polypeptide, such that the amino acid sequence of the encoded polypeptide is altered. In certain embodiments, the polypeptide comprises a functional domain or portion of a functional domain. Systems of the present invention are useful for the introduction of diversity into a targeted position or region of a nucleic acid molecule that encodes a functional domain or portion, such that one or more functions of the domain is enhanced, decreased or otherwise altered. In certain embodiments, systems of the present invention may be used to generate one or more point mutations, deletions, insertions or rearrangements in the functional domain or portion.

In certain embodiments, systems of the present invention may be used to perform saturation mutagenesis on a polypeptide of interest. Saturation mutagenesis is achieved by comprehensively altering the sequence of a polypeptide at one or more amino acid positions such that a plurality of polypeptides that represents every possible amino acid substitution at the one or more positions is generated. This technique permits an unbiased identification of the role that the one or more amino acids plays in the function of the polypeptide and permits determination of which amino acid substitutions result in increased, decreased or otherwise altered functionality of that polypeptide. Due to degeneracy of the genetic code, an alteration in the nucleotide sequence of a template nucleic acid molecule may result in a so-called “silent substitution” that does not alter the peptide sequence of the polypeptide that it encodes. For example, the amino acid leucine is encoded by the codons CTT, CTC, CTG, and CTA. In this case, alteration of the nucleotide located at the third position of a codon that encodes leucine would result in no change in the amino acid sequence of the polypeptide or portion encoded by the template nucleic acid molecule.

The degeneracy of the genetic code simplifies the process of saturation mutagenesis. For example, fewer primers may be required to be present in a degenerate primer set and/or a primer set that contain less degeneracy may be used to achieve saturation mutagenesis of a given polypeptide of interest. An amino acid is encoded by a codon, which consists of three consecutive nucleotides. Thus, there are sixty-four possible nucleotide combinations that may encode an amino acid. However, since many nucleotide substitutions in a given codon are silent and do not alter the amino acid encoded by the codon, not each of the sixty-four possible different codons need be generated to achieve saturation mutagenesis at a particular codon position. In certain embodiments, saturation mutagenesis is achieved at a particular codon position of a template nucleic acid molecule by polymerase-mediated extension with a degenerate primer set that gives rise to a plurality of variant nucleic acid molecules that comprise fewer than the sixty-four possible nucleotide combinations that may encode an amino acid.

In certain embodiments, systems of the present invention are useful for the introduction of diversity into a targeted position or region of a nucleic acid molecule that encodes a polypeptide, such that a given amino acid position of the encoded polypeptide or portion is mutagenized, but the mutagenesis does not reach saturation. For example, in may be desirable to limit the number of possible amino acid alterations at a given position in a polypeptide of interest such that the plurality of variant nucleic acid molecules encode polypeptides of portions that comprise fewer than the twenty possible naturally occurring amino acids at one or more amino acid positions. In certain embodiments, it may be desirable to limit the number of possible amino acid alterations at a given position in a polypeptide of interest such that the plurality of variant nucleic acid molecules encode polypeptides that comprise 2, 3, 4, 5, 6, 7, 8, 9, 10, 11, 12, 13, 14, 15, 16, 17, 18, or 19 amino acid variants at one or more positions.

Since almost all of the twenty naturally occurring amino acids are encoded by at least two codons, the practitioner has some discretion over which particular codon(s) to generate using diversity-targeting systems of the present invention. In some embodiments, a generated plurality of variant nucleic acid molecules comprising diversified position(s) or region(s) are introduced into and/or propagated in a host cell and/or organism in vivo. Certain host cells and/or organisms may prefer one or more particular codons that encode a given amino acid to other codons that encodes the same amino acid. Thus, the practitioner may choose to limit the diversity introduced into the polypeptide-encoding template nucleic acid molecule such that a codon preferred by that host cell and/or organism is exclusively or preferentially introduced over another codon that encodes that same amino acid for purposes of robust propagation. Introducing targeted diversity into one or more positions or regions of a polypeptide-encoding nucleic acid molecule such that the diversified plurality of variant nucleic acid molecules only comprises certain codons may be accomplished through appropriate design of degenerate primer sets. For example, one or more nucleotide positions of at least one degenerate primer set may be non-degenerate such that only one nucleotide residue is present at a particular codon position in the generated plurality of variant nucleic acid molecules. Similarly, one or more nucleotide positions of at least one degenerate primer set may be less than completely degenerate such that fewer than four nucleotide residues are present in a particular codon position in the generated plurality of variant nucleic acid molecules. One of ordinary skill in the art will be aware of the constraints that a given host cell and/or organism places on the nucleotide sequences that encode polypeptides and will be able to choose and/or design degenerate primer sets appropriately in order to avoid undesired variant nucleic acid molecules.

Systems of the present invention may be used to create one or more libraries of nucleic acid molecules that encode polypeptides in which phylogenetic analysis has been used to guide the design of one or more degenerate primer sets such that the diversity targeted to one or more positions or regions of a template nucleic acid molecule is restricted to only positions or regions that encode particular amino acid positions and/or only a subset of the possible amino acids are encoded at those positions. For example, homologous polypeptides encoded by homologous genes, either in different organisms or in the same organism, may differ in their binding activity and/or specificity, their catalytic properties, half-life and/or any other property or characteristic. Such homologous polypeptides may be very similar or identical over a given portion of their lengths, but may be quite divergent at one or more positions or regions, indicating that these positions or regions may play a role in the different properties or characteristics of the polypeptides. Systems of the present invention may be advantageously used to generate a plurality of variant nucleic acid molecules that encode different versions of the homologous polypeptides in which additional diversity has been introduced into the divergent positions or regions. Additionally or alternatively, systems of the present invention may be advantageously used to generate a plurality of variant nucleic acid molecules that encode different versions of the homologous polypeptides in which additional diversity has been introduced into the non-divergent positions or regions. In certain embodiments, the divergent and/or non-divergent positions or regions of the homologous polypeptides are subject to saturation mutagenesis. In certain embodiments, the divergent and/or non-divergent positions or regions of the homologous polypeptides are subject to mutagenesis that does not reach saturation.

Introducing Diversity into a Nucleic Acid Molecule that Does Not Encode a Polypeptide

In certain embodiments, systems of the present invention are used to introduce sequence variation in a targeted position or region of a non-coding nucleic acid molecule. For example, systems of the present invention can be used to introduce or alter a regulatory element that regulates the expression of a polypeptide of interest.

Promoters are non-coding regions generally found just upstream of a gene and regulate expression of that gene, often in response to transcription factors or repressors that bind the promoter. Certain promoters contain discrete promoter elements that function to alter the activity of the promoter, for example in response to various transcription factors or repressors. In some embodiments, systems of the present invention are used to introduce one or more targeted positions or regions of diversity into a promoter or promoter element. Introducing diversity into a promoter or promoter element may be useful, for example, in determining which nucleic acid residue(s) of the promoter or promoter element are important for controlling expression of a polypeptide under control of that promoter or element. In some embodiments, systems of the present invention are used to introduce a heterologous promoter element into a promoter that normally lacks that element. For example, a tissue specific or inducible promoter element may be introduced into an otherwise constitute promoter. A wide variety of promoter elements are known, including but not limited to constitutive elements, inducible elements and tissue-specific elements. One of ordinary skill in the art will be able to determine appropriate promoters or promoter elements into which one or more positions or regions of targeted diversity may be introduced using systems of the present invention. Furthermore, one of ordinary skill in the art will be able to determine the extent of diversity that is to be introduced into the promoter or promoter element, for example, by choosing or controlling the extent of degeneracy of the primers used in the polymerase-mediated extension reaction. In certain embodiments, one or more residues of the promoter or promoter element are subjected to saturation mutagenesis such that a plurality of variant nucleic acid molecules is generated in which every possible nucleotide at those residues is represented.

Introns are non-coding regions found in genomic DNA between regions that encode fragments of a polypeptide. Splicing sites both within and outside the intron regulate its removal after transcription of the genomic DNA into RNA. In certain embodiments, systems of the present invention are used to introduce one or more targeted positions or regions of diversity into an intron or splicing site present in a nucleic acid that encodes a polypeptide of interest. Introducing diversity into an intron or splicing site may be useful, for example, to determine which nucleic acid residues of the intron or splicing site are important for directing removal of the intron and proper splicing of exons. One of ordinary skill in the art will be able to determine appropriate introns or splicing sites into which one or more positions or regions of targeted diversity may be introduced using systems of the present invention. Furthermore, one of ordinary skill in the art will be able to determine the extent of diversity that is to be introduced into the intron or splicing site, for example, by choosing or controlling the extent of degeneracy of the primers used in the polymerase-mediated extension reaction. In certain embodiments, one or more residues of the intron or splicing site are subjected to saturation mutagenesis such that a plurality of variant nucleic acid molecules is generated in which every possible nucleotide at those residues is represented.

In some situations, not only are the introns spliced out upon transcribing genomic DNA into RNA, but one or more exons may also be spliced out in a process known as alternative splicing. The degree and nature of alternative splicing depends on, among other factors, cell type, tissue type, developmental stage, and/or environmental conditions of the organism or cell. Whether certain exons are spliced out of a given transcript depends at least in part on the nucleotide sequences of exons and/or introns of those genes. Thus, by introducing diversity into targeted positions or regions of the exons and/or introns of alternatively spliced genes, systems of the present invention provide a useful tool for studying the nucleotide sequences that are important for determining alternative splicing patterns.

In certain embodiments, systems of the present invention are used to introduce one or more targeted positions or regions of diversity into a regulatory element located in the 3′ or 5′ UTR of a particular mRNA molecule. Regulatory element may regulate, for example, the stability or translation of that mRNA. One of ordinary skill in the art will be able to determine appropriate UTR regulatory elements into which one or more positions or regions of targeted diversity may be introduced using systems of the present invention. Furthermore, one of ordinary skill in the art will be able to determine the extent of diversity that is to be introduced into the UTR regulatory element, for example, by choosing or controlling the extent of degeneracy of the primers used in the polymerase-mediated extension reaction. In certain embodiments, one or more residues of the UTR element are subjected to saturation mutagenesis such that a plurality of variant nucleic acid molecules is generated in which every possible nucleotide at those residues is represented.

Numerous other regulatory elements are known in the art and one of ordinary skill in the art will be able to determine appropriate and/or desirable regulatory elements into which one or more targeted positions or regions of diversity is to be introduced according to teachings of the present invention.

Introducing Sequence Diversity via Polymerase-Mediated Extension

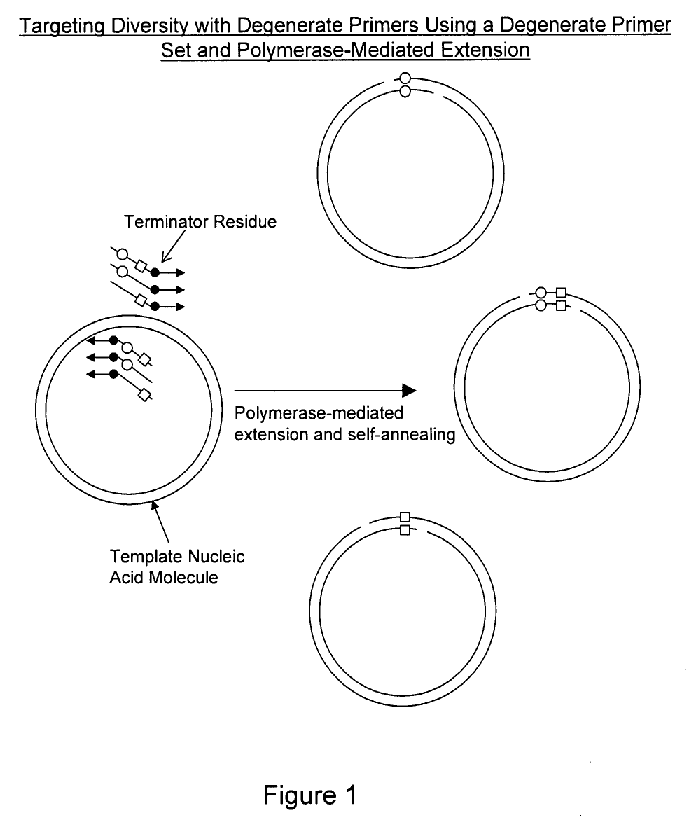

In certain embodiments, sequence diversity is introduced into a nucleic acid molecule by subjecting a template nucleic acid molecule to a polymerase-mediated extension reaction in which at least one of the primers of a primer pair contains one or more terminator nucleotides and/or one or more terminator structures that do not serve as templates for the polymerase used in the polymerase-mediated extension reaction. According to such embodiments, a nucleic acid molecule generated from the polymerase-mediated extension reactions contains at least one 5′ overhang resulting from termination of polymerization at the terminator nucleotide and/or terminator structure. One embodiment of introducing sequence diversity using a degenerate primer set is shown in FIG. 1.

As non-limiting examples, a terminator nucleotide may be a ribonucleotide or a 2′-O-methyl nucleotide. One of ordinary skill in the art will be aware of other nucleotides or nucleotide analogs that may be used as terminator residues in accordance with systems of the present invention. Ribonucleotide terminator nucleotides may be used in polymerase-mediated extension reactions with DNA polymerases that are not able to use ribonucleotides as templates under at least one set of polymerization conditions. For example, Vent_{R}® and Vent_{R}® (exo⁻) (Vent_{R}® and Vent_{R}® (exo⁻) are enzymes purified from strains of E. coli that carry either the DNA polymerase gene isolated from the archaea Thermococcus litoralis or a version of the gene that has been genetically engineered to eliminate the 3′ to 5′ proofreading exonuclease activity associated with Vent_{R}® DNA polymerase) do not use ribonucleotide bases as a template. Tth and Taq polymerases, by contrast, are reported to be able to replicate ribonucleotides under at least one set of polymerization conditions (Myers et al., Biochem. 6:7661, 1991), as, of course, are reverse transcriptases. One of ordinary skill in the art will have the ability to determine whether other DNA polymerases are able to use ribonucleotide residues as templates according to standard techniques without undue experimentation.

In certain embodiments, a terminator nucleotide may be used as a template by a different polymerase and/or the same polymerase under a different set of reaction conditions. For example, certain polymerases are capable of using ribonucleotide residues as templates under certain reaction conditions. One of ordinary skill in the art will be able to determine whether a given polymerase may use a particular terminator nucleotide as a template under a given set of polymerization conditions according to standard techniques known in the art. In certain embodiments, one or both primers contain more than one terminator nucleotide. For example, a primer may contain 1, 2, 3, 4, 5, 6, 7, 8, 9, 10 or more terminator nucleotides. In certain embodiments, two or more terminator nucleotides are directly adjacent to one another. In certain embodiments, two or more terminator nucleotides are separated from each other by one or more non-terminator nucleotides. In certain embodiments, one or both primers contain more than one type of terminator residue.

In some embodiments, at least one of the primers used in the polymerase-mediated extension reaction contains a terminator structure that does not serve as a replication template for the polymerase used in the extension reaction. In certain embodiments, a terminator structure is an abasic site. In certain embodiments, both primers used in the polymerase-mediated extension contain a terminator nucleotide and/or a terminator structure.

In certain embodiments, the polymerase-mediated extension reaction is performed according to one or more of the methods disclosed in U.S. Pat. No. 6,358,712, incorporated herein by reference in its entirety.

Any polymerase that is able to copy at least one of the nucleotides present in the primers may be used in the polymerase-mediated extension reaction. In certain embodiments, the polymerase-mediated extension reaction is PCR. In certain aspects of this embodiment, a thermostable polymerase may be used such that a template nucleic acid molecule may be extended by PCR in one reaction chamber. As non-limiting examples, thermostable polymerases derived from Thermus aquaticus (“Taq”), Pyrococcus furiousus (“Pfu”), Thermus thermophilus (“Tth”), Thermococcus gorgonarius (“Tgo”), Thermus flavusi (“Tfl”), Thermus brockianus (“Tbr”), Thermococcus litoralis (“Vent”) and Bacillus stearothermophilus (“Bst”) may be used under one or more polymerization conditions. One of ordinary skill in the art will be aware of other appropriate polymerases that may be used in the polymerase-mediated extension, as well as the appropriate enzymatic conditions that facilitate and/or allow polymerization.

In certain embodiments, at least one primer of the primer pair contains a first portion that anneals with a template nucleic acid molecule under at least one set of conditions, a second portion that does not serve as a template for at least one polymerase under at least one set of polymerization conditions, and a third portion may or may not anneal to the template nucleic acid molecule. One of ordinary skill in the art will understand that the first portion of the primer need not be perfectly complementary to a template nucleic acid molecule in order for annealing to occur. For example, the first portion may contain one or more mismatches when annealed to a template nucleic acid molecule so long as the remaining sequence of the first portion provides sufficient complementarity to permit annealing. In certain embodiments, it is not necessary that the 3′-most nucleotide of the first portion be complementary to a template nucleic acid so long as the first portion is able to anneal to the template nucleic acid and the non-paired nucleotide is able to serve as a priming site for polymerase-mediated extension. Furthermore, those of ordinary skill in the art will also appreciate that if the DNA polymerase being employed includes a 3′ to 5′ exonuclease activity, the 3′-most residue in the primer that does not anneal to a template nucleic acid molecule may be chewed back to a point in the primer where the 3′-most nucleotide is complementary to the template nucleic acid molecule.

In certain embodiments, one or both primers comprise degenerate position(s) that are located 5′ to the terminator nucleotide or structure. According to such embodiments, after polymerase-mediated extension of a template nucleic acid molecule, degenerate position(s) will be located in the single-stranded 5′ overhang of the polymerase-mediated extension products (see for example, the embodiment shown in FIG. 1). In certain embodiments, a region 5′ to the terminator nucleotide or structure that contains one or more degenerate positions is substantially complementary to a template nucleic acid molecule such that the region is capable of annealing to a template nucleic acid molecule under at least one set of polymerization conditions. For example, a region 5′ to the terminator nucleotide or structure may contain only one or only a few degenerate positions in order to introduce a minimal or controlled amount of sequence diversity into the targeted region of the nucleic acid molecule.

In certain embodiments, a region 5′ to the terminator nucleotide or structure that contains one or more degenerate positions is not substantially complementary to a template nucleic acid molecule such that the region does not anneal to the template nucleic acid molecule under at least one set of polymerization conditions. According to such embodiments, the region 3′ to the terminator nucleotide or structure is sufficiently complementary to a template nucleic acid molecule over a sufficient length to permit the primer to anneal to the template nucleic acid molecule under a given set of polymerization conditions. For example, the region 5′ to the terminator nucleotide or structure may be substantially or completely heterologous to a template nucleic acid molecule in order to introduce a relatively large amount of diversity into the targeted region of the nucleic acid molecule.

In certain embodiments, a region 5′ to the terminator nucleotide or structure that contains one or more degenerate positions is of intermediate complementarity to a template nucleic acid molecule, such that the 5′ region anneals to the template nucleic acid molecule under one set of polymerization conditions but does not anneal under a different set of polymerization conditions.

In certain embodiments, degenerate position(s) of one or both primers are located 3′ to the terminator nucleotide or structure, provided that the degenerate position(s) do not prevent the primer from annealing to a template nucleic acid molecule and priming extension. According to such embodiments, after polymerase-mediated extension, degenerate position(s) will be located in the double-stranded region of the extension products. In certain embodiments, degenerate position(s) of one or both primers are located both 5′ to the terminator nucleotide or structure as well as 3′ to the terminator nucleotide or structure. Such embodiments permit a greater amount of sequence diversity to be introduced into a nucleic acid molecule in a single reaction.

In certain embodiments, terminator PCR is used to introduce two or more targeted positions or regions of sequence diversity into a nucleic acid molecule (for example, see the embodiment shown in FIG. 2).

It may be desirable under certain circumstances to limit the number of degenerate positions and/or the number of different residues representing each position of degeneracy. For example, it may be desirable to provide primers that contain one or more degenerate positions, wherein the degenerate position(s) are represented by fewer than the four naturally occurring nucleotides. In other circumstances, it may be desirable to maximize the number of degenerate positions and/or the number of different residues representing each position of degeneracy. For example, it may be desirable to provide primers that contain one or more degenerate positions, wherein the degenerate position(s) are represented by all four naturally occurring nucleotides. One of ordinary skill in the art will be able to choose the number of degenerate positions to be used in one or both primers. Furthermore, one of ordinary skill in the art will be able to choose the number of different residues representing each position of degeneracy.

Introducing Diversity with Cleavable/Removable Residues

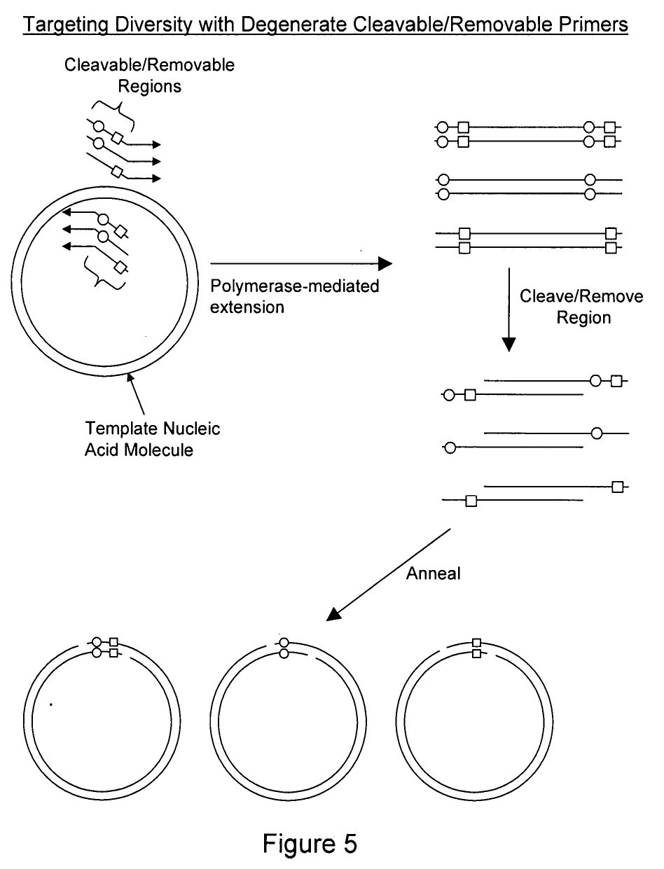

In certain embodiments, targeted sequence diversity is introduced into a nucleic acid molecule by subjecting a template nucleic acid molecule to a polymerase-mediated extension reaction in which at least one of the primers in a degenerate primer set contains one or more nucleotide residues that are capable of being cleaved or removed subsequent to the polymerase-mediated extension reaction (for example, see the embodiment shown in FIG. 5).

For example, one or both of the primers used in the polymerase-mediated extension reaction may contain one or more ribonucleotide residues. After polymerase-mediated extension with a polymerase that is able to use ribonucleotide residues as templates, the ribonucleotide residue(s) may be cleaved or removed from the extension product by any of a number of methods well known in the art. For example, ribonucleotide residues present in the extension product may be cleaved or removed by exposure to elevated pH (e.g., treatment with a base such as sodium hydroxide). Any other treatment that removes ribonucleotide residues without disturbing DNA residues (e.g., exposure to RNase, etc.) could alternatively be employed at this step. One of ordinary skill in the art will be aware of other known treatments that cleave or remove ribonucleotide residues such that the treated molecule may be used in accordance with systems and methods disclosed herein.

Other cleavable or removable residues and methods for cleaving or removing them are encompassed by the present invention. Those of ordinary skill in the art will be aware of and will be able to utilize other such cleavable or removable residues and methods.

In certain embodiments, at least one member of a degenerate primer set contains a region that is capable of being cleaved or removed, which region extends to the 5′ end of the primer(s). Removal of the region(s) results in a plurality of variant nucleic acid molecules, each member of which is partially double-stranded and contains at least one terminal 3′ single-stranded overhang.

In certain embodiments, at least one primer of the primer pair contains a first portion that anneals with a template nucleic acid molecule under at least one set of conditions, and a second portion that is positioned 5′ to the first portion, which second portion may or may not anneal to the template nucleic acid molecule. One of ordinary skill in the art will understand that the first portion of the primer need not be perfectly complementary to a template nucleic acid molecule in order for annealing to occur. For example, the first portion may contain one or more mismatches when annealed to a template nucleic acid molecule so long as the remaining sequence of the first portion provides sufficient complementarity to permit annealing. In certain embodiments, it is not necessary that the 3′-most nucleotide of the first portion be complementary to a template nucleic acid so long as the first portion is able to anneal to the template nucleic acid and the non-paired nucleotide is able to serve as a priming site for polymerase-mediated extension. Furthermore, those of ordinary skill in the art will also appreciate that if the DNA polymerase being employed includes a 3′ to 5′ exonuclease activity, the 3′-most residue in the primer that does not anneal to a template nucleic acid molecule may be chewed back to a point in the primer where the 3′-most nucleotide is complementary to the template nucleic acid molecule.

In certain embodiments, residues that may be cleaved or removed after polymerization are located exclusively in the second portion that may or may not anneal to a template nucleic acid molecule. In certain embodiments, both the first and second portions of the primer(s) contain residues that may be cleaved or removed subsequent to polymerization.

In certain embodiments, degenerate position(s) of one or both primers are located in the region that is capable of being cleaved or removed after extension. According to such embodiments, after polymerase-mediated extension and cleavage or removal of the region, degenerate position(s) will be located in the 3′ single-stranded region of the extension products. In certain embodiments, the region capable of being cleaved or removed that contains one or more degenerate positions is substantially complementary to a template nucleic acid molecule such that the region is capable of annealing to the template nucleic acid molecule under at least one set of polymerization conditions. For example, a region capable of being cleaved or removed may contain only one or only a few degenerate positions in order to introduce a minimal or controlled amount of sequence diversity into the targeted region of a nucleic acid molecule.

Additionally or alternatively, degenerate position(s) of one or both primers are located 3′ to the region to that is capable of being cleaved or removed after extension, provided that the degenerate position(s) do not prevent the primer from annealing to a template nucleic acid molecule and priming extension. According to such embodiments, degenerate position(s) will be located in the double-stranded region of the extension products. In certain embodiments, degenerate position(s) of one or both primers are located both in the region to that is capable of being cleaved or removed as well as in the region to that is not capable of being cleaved or removed. Such embodiments permit a greater amount of sequence diversity to be introduced into a nucleic acid molecule in a single reaction.

Combination with Recipient Nucleic Acid Molecules

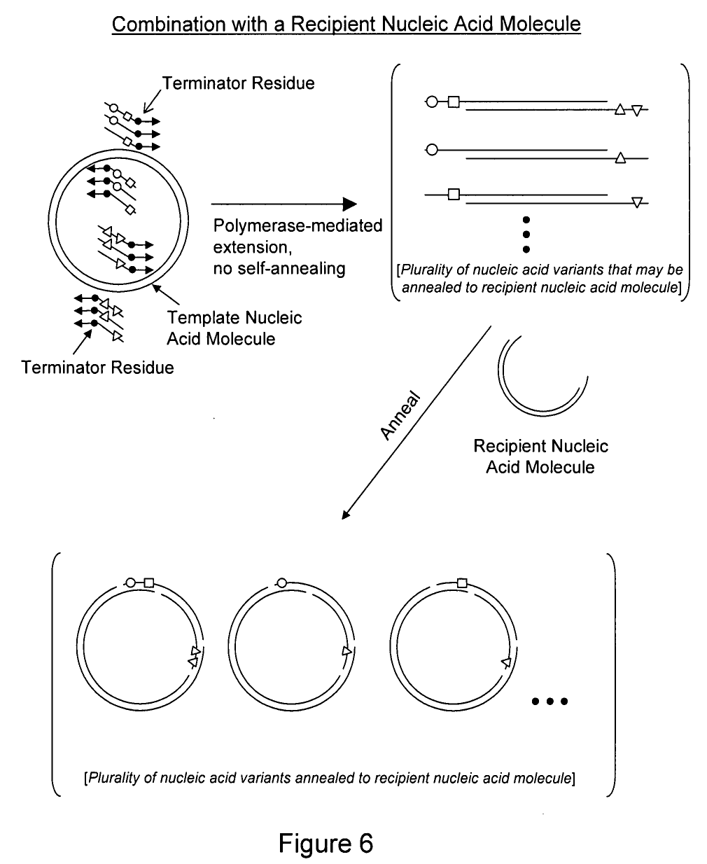

In certain embodiments, systems of the present invention are used to introduce one or more targeted positions or regions of diversity into a nucleic acid molecule to generate a plurality of variant nucleic acid molecules, which plurality is subsequently combined with one or more recipient nucleic acid molecules (for example, see the embodiment shown in FIG. 6).

In certain embodiments, a plurality of variant nucleic acid molecules is combined with one or more recipient nucleic acid molecules to generate a circular nucleic acid molecule. For example, members of the plurality of variant nucleic acid molecules may contain one or more single-stranded termini, which single-stranded termini are not able to anneal to each other under at least one set of annealing conditions. The plurality of variant nucleic acid molecules may then be annealed with one or more recipient nucleic acid molecules that also contain one or more single-stranded termini, which single-stranded termini are not able to anneal to each other under at least one set of annealing conditions, but one or both of which are able to anneal to one or both of the single-stranded termini of the plurality of variant nucleic acid molecules. Upon joining, a circular nucleic acid molecule is generated that contains one or more targeted positions or regions of diversity.

One of ordinary skill in the art will understand that the single-stranded termini of the plurality of variant nucleic acid molecules need not be perfectly complementary to the single-stranded termini of the recipient nucleic acid molecule, so long as the termini are able to anneal to each other under at least one set of annealing conditions. If such single-stranded termini are not perfectly complementary, the single-stranded termini will form a double-stranded region upon annealing that will necessarily contain one or more mismatched base pairs. Such mismatched base pairs may be resolved by methods known to those skilled in the art. For example, mismatches may be resolved by use of an in vitro repair system or by introducing the combined nucleic acid molecule into a cell that is capable of resolving mismatches. In certain embodiments, mismatched base pairs created upon combining one or more variant nucleic acid molecules with one or more recipient nucleic acid molecules may be exploited to reduce the number of primers required to be present in a degenerate primer set to achieve a desired level of degeneracy in one or more targeted regions of a nucleic acid molecule.

In certain embodiments, a recipient nucleic acid molecule is generated by one or more traditional methods including, for example, cleaving a nucleic acid molecule with one or more restriction endonucleases. In certain embodiments, cleavage with one or more restriction endonucleases results in a recipient nucleic acid molecule that contains terminal single-stranded regions. In certain embodiments, such single-stranded termini of the recipient nucleic acid molecule do not anneal to each other under at least one set of annealing conditions, but do anneal to one or both single stranded termini of members of the plurality of variant nucleic acid molecules.

In certain embodiments, a recipient nucleic acid molecule is generated by annealing and/or ligating one or more adapter oligonucleotides to a linear nucleic acid molecule. In certain embodiments, the adapter oligonucleotides form recipient nucleic acid molecule that contain one or more single-stranded termini. In certain embodiments, single-stranded termini of the recipient nucleic acid molecule generated by annealing and/or ligating one or more adapter oligonucleotides do not anneal to each other under at least one set of annealing conditions, but do anneal to one or both single stranded termini of members of the plurality of variant nucleic acid molecules.

In certain embodiments, a recipient nucleic acid molecule is generated according to any of the methods disclosed in U.S. patent application Ser. No. 11/271,561, incorporated herein by reference in its entirety. For example, a recipient nucleic acid molecule may be generated by the process of gap amplification. Gap amplification comprises subjecting a nucleic acid molecule to one or more polymerase-mediated extensions such that one or more linear recipient nucleic acid molecules are generated. Gap amplification may be performed on a circular nucleic acid molecule. Alternatively, gap amplification may be performed on a linear nucleic acid molecule. In certain embodiments, gap amplification is performed according to one or more of the methods disclosed in U.S. Pat. No. 6,358,712, incorporated herein by reference in its entirety. In some embodiments, gap amplification is performed according to one or more of the methods disclosed in U.S. patent application Ser. No. 10/383,135, incorporated herein by reference in its entirety.

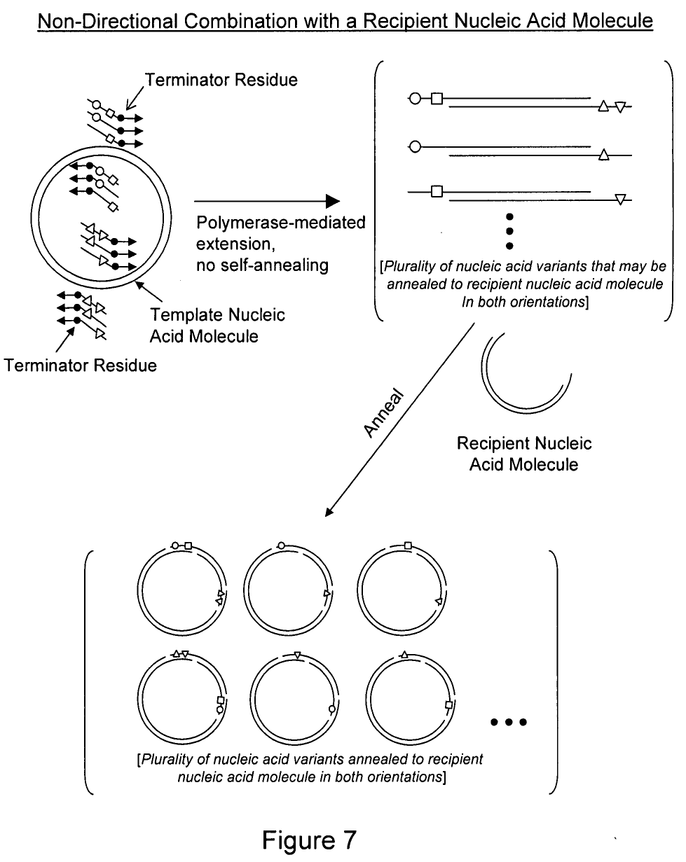

In certain embodiments, members of the plurality of variant nucleic acid molecules are joined with a recipient nucleic acid molecule in a directional manner, such that members of the plurality are joined to the recipient nucleic acid molecule in only one orientation. In certain embodiments, members of the plurality of variant nucleic acid molecules are joined with a recipient nucleic acid molecule in a non-directional manner, such that members of the plurality are joined to the recipient nucleic acid molecule in both orientations (for example, see the embodiment shown in FIG. 7).

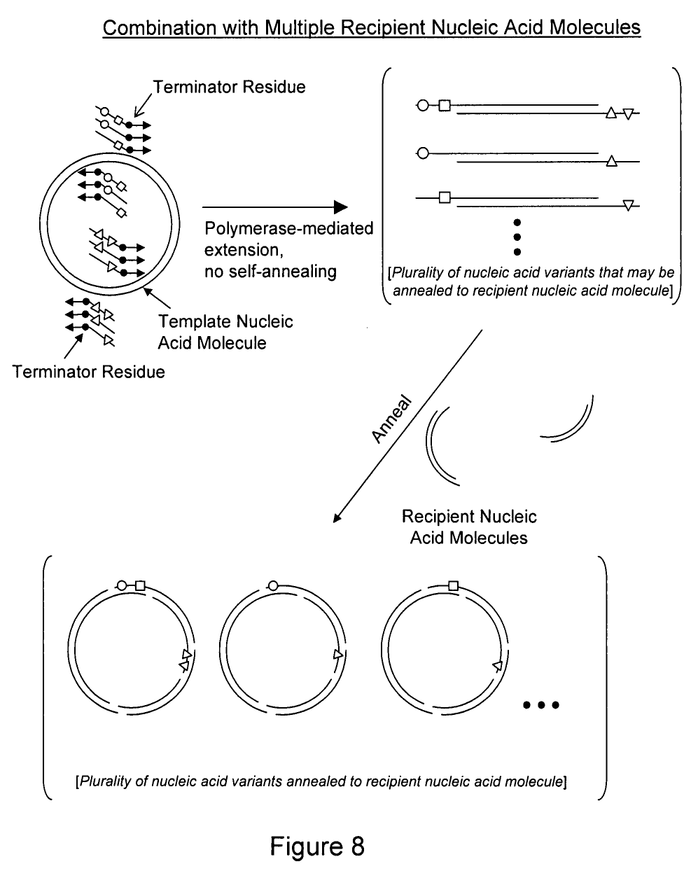

In certain embodiments, two or more recipient nucleic acid molecules are joined to a plurality of variant nucleic acid molecules (for example, see the embodiment shown in FIG. 8). For example, a plurality of variant nucleic acid molecules may be generated containing one or more positions or regions of targeted diversity, which plurality is combined with two or more recipient nucleic acid molecules. In certain embodiments, individual recipient nucleic acid molecules are positioned adjacent to each other in the combined nucleic acid molecule.

In certain embodiments, a recipient nucleic acid molecule is joined to two or more pluralities of variant nucleic acid molecules. For example, a first and second plurality of variant nucleic acid molecules may be generated, each plurality containing one or more positions or regions of targeted diversity, which first and second pluralities are combined with a recipient nucleic acid molecule. In certain embodiments, individual members of the first and second plurality are positioned adjacent to each other in the combined nucleic acid molecule.

In certain embodiments, the first and second pluralities are combined with two or more recipient nucleic acid molecules. For example, the first and second plurality may be combined with two recipient nucleic acid molecules. In certain embodiments, individual members of the first and second plurality are positioned adjacent to each other in the combined nucleic acid molecule and/or individual recipient nucleic acid molecules are positioned adjacent to each other in the combined nucleic acid molecule. Additionally or alternatively, individual members of the first and second plurality are separated from each other in the combined nucleic acid molecule by one or more recipient nucleic acid molecules.

Kits

Reagents useful for the practice of the present invention may desirably be provided together, assembled in a kit. In certain embodiments, kits include reagents useful for generating a plurality of variant nucleic acid molecules according to any of the systems described herein. For example, kits may include reagents useful for generating a plurality of variant nucleic acid molecules by using polymerase-mediated extension. In certain embodiments, the polymerase-mediated extension reaction is PCR. Additionally or alternatively, kits may include reagents useful for generating a plurality of variant nucleic acid molecules by using PCR with cleavable/removable residues.

In certain embodiments, kits include one or more recipient nucleic acid molecules that may be combined with one or more pluralities of variant nucleic acid molecules. In certain embodiments, kits include ligase for ligating annealed variant nucleic acid molecules and/or recipient nucleic acid molecules. In certain embodiments, kits include host cells for transforming and/or propagating a nucleic acid molecule produced in accordance with any of the methods described herein.

The foregoing description is to be understood as being representative only and is not intended to be limiting. Alternative methods and materials for implementing the invention and also additional applications will be apparent to one of skill in the art, and are intended to be included within the accompanying claims.

What is claimed is:

1. A method of introducing a sequence change into a nucleic acid molecule comprising the steps of:
providing a template nucleic acid molecule;
providing at least one primer pair, wherein each primer of the primer pair comprises
1) a first portion that anneals to the template nucleic acid molecule,
2) a second portion that does not serve as a template for at least one polymerase under at least one set of polymerization conditions, and
3) a third portion;
wherein the third portions of each primer of the primer of the primer pair are capable of annealing to each other under at least one set of annealing conditions; and
wherein the sequence of at least one primer of the primer pair comprises at least one residue that differs from the sequence of the template nucleic acid molecule;
extending the primer pair against the template nucleic acid molecule in a polymerase-mediated extension reaction to generate a linear nucleic acid molecule comprising a central double-stranded portion and two terminal single-stranded portions, each single-stranded portion comprising the third portion of one primer of the primer pair; and
annealing the single-stranded portions of the linear nucleic acid molecule to generate a first product nucleic acid molecule that comprises a sequence different from the sequence of the template nucleic acid molecule.

2. The method of claim 1, wherein the template nucleic acid molecule is single-stranded.

3. The method of claim 1, wherein the template nucleic acid molecule is double-stranded.

4. The method of claim 1, wherein the second portion of at least one primer of the primer pair comprises a ribonucleotide.

5. The method of claim 1, wherein the second portion of at least one primer of the primer pair comprises a plurality of ribonucleotides.

6. The method of claim 5, wherein at least two ribonucleotides of the plurality of ribonucleotides are directly adjacent to each other.

7. The method of claim 1, wherein the second portion of at least one primer of the primer pair comprises a 2′-O-methyl residue.

8. The method of claim 1, wherein the second portion of at least one primer of the primer pair comprises a plurality of 2′-O-methyl residues.

9. The method of claim 8, wherein at least two 2′-O-methyl residues of the plurality of 2′-O-methyl residues are directly adjacent to each other.

10. The method of claim 1, wherein the second portion of at least one primer of the primer pair comprises a terminator structure.

11. The method of claim 10, wherein the terminator structure comprises an abasic residue.

12. The method of claim 1, wherein the third portion of at least one of the primers does not anneal to template nucleic acid molecule.

13. The method of claim 1, wherein the at least one residue that differs from the sequence of the template nucleic acid molecule is located in the first portion of the primer.

14. The method of claim 1, wherein the at least one residue that differs from the sequence of the template nucleic acid molecule is located in the third portion of the primer.

15. The method of claim 1, wherein at least one primer of the primer pair further comprises one or more additional residues that do not correspond in position to any residue present in the template nucleic acid molecule, such that the first product nucleic acid molecule contains an insertion of one or more residues as compared to the template nucleic acid molecule.

16. The method of claim 1, wherein at least one primer of the primer pair lacks one or more residues that are present in the template nucleic acid molecule, such that the first product nucleic acid molecule contains a deletion of one or more residues as compared to the template nucleic acid molecule.

17. The method of claim 16, wherein the primer of the primer pair that lacks one or more residues further comprises one or more additional residues that do not correspond in position to any residue present in the template nucleic acid molecule, such that the first product nucleic acid molecule contains both an insertion of one or more residues and a deletion of one or more residues as compared to the template nucleic acid molecule.

18. The method of claim 1, wherein the first product nucleic acid molecule is used as template nucleic acid molecule to generate a second product nucleic acid molecule, further comprising the steps of:
providing at least one additional primer pair, wherein each primer of the additional primer pair comprises
1) a first portion that anneals to the first product nucleic acid molecule,
2) a second portion that does not serve as a template for at least one polymerase under at least one set of polymerization conditions, and
3) a third portion;
wherein the third portions of each primer of the additional primer pair are capable of annealing to each other under at least one set of annealing conditions; and
wherein the sequence of at least one primer of the additional primer pair comprises at least one residue that differs from the sequence of the first product nucleic acid molecule;
extending the additional primer pair against the first product nucleic acid molecule to generate a linear nucleic acid molecule comprising a central double-stranded portion and two terminal single-stranded portions, each single-stranded portion comprising the third portion of one primer of the additional primer pair;
annealing the single-stranded portions of the linear nucleic acid molecule to generate a second product nucleic acid molecule that comprises a sequence different from the sequence of the first product nucleic acid molecule.

19. The method of claim 1, wherein the primer residue that differs from the sequence of the template nucleic acid molecule is fully degenerate.

21. The method of claim 1, wherein the primer residue that differs from the sequence of the template nucleic acid molecule is partially degenerate.

22. The method of claim 1, wherein the first product nucleic acid molecule encodes a polypeptide.

23. A method of introducing a sequence change into a nucleic acid molecule comprising the steps of:
providing a template nucleic acid molecule;
providing at least one primer pair, wherein each primer of the primer pair comprises
1) a first portion that is capable of annealing to the template nucleic acid molecule; and
2) a second portion located at the 5′ end of the primer that renders the primer susceptible to cleavage under at least one set of conditions under which DNA of identical sequence is not susceptible to cleavage;
wherein the second portions of each primer of the primer pair are capable of annealing to each other under at least one set of annealing conditions; and
wherein the sequence of at least one primer of the primer pair comprises at least one residue that differs from the sequence of the template nucleic acid molecule;
extending the primer pair against the template nucleic acid molecule in a polymerase-mediated extension reaction to generate a linear double-stranded nucleic acid molecule;
subjecting the linear double-stranded nucleic acid molecule to cleavage conditions such that the nucleotide residues susceptible to cleavage are removed, resulting in a partially double-stranded nucleic acid molecule that contains at least one single-stranded portion; and
annealing the single-stranded portions of the partially double-stranded nucleic acid molecule to generate a first product nucleic acid molecule that comprises a sequence different from the sequence of the template nucleic acid molecule.

24. The method of claim 23, wherein the template nucleic acid molecule is single-stranded.

25. The method of claim 23, wherein the template nucleic acid molecule is double-stranded.

26. The method of claim 23, wherein the second portion comprises a ribonucleotide.

27. The method of claim 23, wherein the second portion comprises a plurality of ribonucleotides.

28. The method of claim 26 or 27, wherein set of the subjecting comprises subjecting the product nucleic acid molecule to pH sufficient to effect cleavage of the ribonucleotide residue or residues.

29. The method of claim 28, wherein the step of subjecting comprises subjecting the product nucleic acid molecule to sodium hydroxide.

30. The method of claim 26 or 27, wherein the step of subjecting comprises subjecting the product nucleic acid molecule to RNase that removes the ribonucleotide residues without removing deoxyribonucleotide residues.

31. The method of claim 23, wherein the second portion of at least one of the primers does not anneal to the template nucleic acid molecule.

32. The method of claim 23, wherein the at least one residue that differs from the sequence of the template nucleic acid molecule is located in the first portion of the primer.

33. The method of claim 23, wherein the at least one residue that differs from the sequence of the template nucleic acid molecule is located in the second portion of the primer.

34. The method of claim 23, wherein at least one primer of the primer pair further comprises one or more additional residues that do not correspond in position to any residue present in the template nucleic acid molecule, such that the first product nucleic acid molecule contains an insertion of one or more residues as compared to the template nucleic acid molecule.

35. The method of claim 23, wherein at least one primer of the primer pair lacks one or more residues that is present in the template nucleic acid molecule, such that the first product nucleic acid molecule contains a deletion of one or more residues as compared to the template nucleic acid molecule.

36. The method of claim 35, wherein the primer that lacks one or more residues further comprises one or more additional residues that do not correspond in position to any residue present in the template nucleic acid molecule, such that the first product nucleic acid molecule contains both an insertion of one or more residues and a deletion of one or more residues as compared to the template nucleic acid molecule.

37. The method of claim 23, wherein the at least one residue that differs from the sequence of the template nucleic acid molecule is fully degenerate.

38. The method of claim 23, wherein the at least one residue that differs from the sequence of the template nucleic acid molecule is partially degenerate.

39. The method of claim 23, wherein the first product nucleic acid molecule encodes a polypeptide.

40. A method of introducing a sequence change into a nucleic acid molecule comprising the steps of:
providing a template nucleic acid molecule;
providing at least one primer pair, wherein each primer of the primer pair comprises
1) a first portion that anneals to the template nucleic acid molecule,
2) a second portion that does not serve as a template for at least one polymerase under at least one set of polymerization conditions, and
3) a third portion;
wherein the sequence of at least one primer of the primer pair comprises at least one residue that differs from the sequence of the template nucleic acid molecule;
extending the primer pair against the template nucleic acid molecule in a polymerase-mediated extension reaction to generate a linear nucleic acid molecule comprising a central double-stranded portion and two terminal single-stranded portions, each single-stranded portion comprising the third portion of one primer of the primer pair;
providing a recipient nucleic acid molecule comprising a central double-stranded portion and two terminal single-stranded portions, which terminal single-stranded portions are capable of annealing to the terminal single-stranded portions of the generated linear nucleic acid molecule under at least one set of annealing conditions; and
combining the generated linear nucleic acid molecule and the recipient nucleic acid molecule under a set of conditions that permits annealing of the terminal single-stranded portions of the generated linear nucleic acid molecule with the terminal single-stranded portions of the recipient nucleic acid molecule.

41. A method of introducing a sequence change into a nucleic acid molecule comprising the steps of:
providing a template nucleic acid molecule;
providing at least one primer pair, wherein each primer of the primer pair comprises
1) a first portion capable of annealing to the template nucleic acid molecule; and
2) a second portion located at the 5′ end of the primer that renders the primer susceptible to cleavage under at least one set of conditions under which DNA of identical sequence is not susceptible to cleavage;
wherein the sequence of at least one primer of the primer pair comprises at least one residue that differs from the sequence of the template nucleic acid molecule;
extending the primer pair against the template nucleic acid molecule in a polymerase-mediated extension reaction to generate a linear double-stranded nucleic acid molecule;
subjecting the linear double-stranded nucleic acid molecule to cleavage conditions such that the nucleotide residues susceptible to cleavage are removed to generate a partially double-stranded nucleic acid molecule comprising at least one terminal single-stranded portion;
providing a recipient nucleic acid molecule comprising a central double-stranded portion and two terminal single-stranded portions, which terminal single-stranded portions are capable of annealing to the terminal single-stranded portions of the generated linear nucleic acid molecule under at least one set of annealing conditions; and
combining the generated linear nucleic acid molecule and the recipient nucleic acid molecule under a set of conditions that permits annealing of the terminal single-stranded portions of the generated linear nucleic acid molecule with the single-stranded portions of the recipient nucleic acid molecule.
